(19) United States
(12) Patent Application Publication
(10) Pub. No.: US 2026/0234685 A1
(43) Pub. Date: Aug. 13, 2026

(54) DNA-Dependent Synthesis of RNA by DNA Polymerase Theta Variants

(71) Applicant: Thomas Jefferson University, Philadelphia, PA (US)

(72) Inventor: Richard T Pomerantz, Philadelphia, PA (US)

(21) Appl. No.: 19/471,184

(22) PCT Filed: Mar. 29, 2024
(86) PCT No.: PCT/US2024/022276

§ 371 Date: Sep. 30, 2025

Related U.S. Application Data
(60) Provisional application No. 63/493,194, filed on Mar. 30, 2023.

(51) Int. Cl. C12P 19/34; C12N 9/12

(52) U.S. Cl. CPC C12P 19/34; C12N 9/1252; C12Y 207/07007

(57) ABSTRACT

Synthetic RNA composed of canonical and modified ribonucleotides is highly effective for RNA antisense therapeutics, RNA based genome engineering applications and RNA based vaccines. Yet, synthesis of sequence-specific synthetic RNA using phosphoramidite chemistry is highly inefficient and expensive, especially for relatively long (>95 nt) RNA with chemical modifications. The present invention relates to enzymatic DNA-dependent synthesis of sequence-specific synthetic RNA with and without chemical modifications using A-family DNA polymerase variants and the use of such chemically modified RNA for applications such as RNA-based genome engineering and therapeutics.

### CROSS-REFERENCE TO RELATED APPLICATIONS

This application claims the benefit of U.S. Provisional Patent Application No. 63/493,194 filed Mar. 30, 2023, which is incorporated herein by reference in its entirety.

### REFERENCE TO A “SEQUENCE LISTING” SUBMITTED AS AN XML FILE

The present application hereby incorporates by reference the entire contents of the XML file named “205961-0063-00WO_SequenceListing.xml” in XML format, which was created on Mar. 27, 2024, and is 32,360 bytes in size.

### BACKGROUND OF THE INVENTION

The ability to synthesize long (>1,000 nucleotide (nt)) mRNA molecules with modified ribonucleotides (i.e., pseudouridine) for vaccine production has been optimized by developing modified bacteriophage RNA polymerases (RNAPs) (Elkhalifa, D., et al., 2022, Biomed Pharmacother, 145:112385; Chelliserrykattil, J. et al., 2004, Nat Biotechnol, 22:1155-1160; Rosa, S. S., et al., 2021, Vaccine, 39:2190-2200). In contrast, the synthesis of shorter (i.e., ~16-150 nt) RNA oligonucleotides containing site-specific chemical modifications such as those used for anti-sense RNA therapeutics (i.e., gapmer and siRNA) and CRISPR genome engineering (sgRNA) remains inefficient, resulting in low yields, and is expensive, especially for kilogram production of anti-sense RNA therapeutics and therapeutic grade sgRNA (Molina, A. G., et al., 2019, Curr Protoc Nucleic Acid Chem, 77: e82; Catani, M., et al., 2020, Biotechnol J, 15: e1900226; Roy, S., et al., 2013, Molecules, 18:14268-14284; Crooke, S. T., et al., 2021, J Biol Chem, 296:100416; Crooke, S. T., et al., 2021, Nat Rev Drug Discov, 20:427-453; Glazier, D. A., et al., 2020, Bioconjug Chem, 31:1213-1233). Such RNA oligonucleotides are synthesized using phosphoramidite chemistry which requires multiple steps per nucleotide addition, generates high levels of toxic waste, and is unable to generate relatively long (>50 nt) RNA at low cost and high yields, posing as a major obstacle for the development and manufacturing of synthetic RNA for genome engineering and other applications.

There is a need in the art for compositions and methods for synthesizing and using oligonucleotides. The present invention satisfies this unmet need.

### SUMMARY OF THE INVENTION

In one aspect, the disclosure provides methods of synthesizing a sequence-specific oligonucleotide, the method comprising the steps of: providing a DNA template in a solution; contacting the nucleic acid template in the solution with a primer; and contacting the nucleic acid primer-template in the solution with an A-family DNA polymerase mutant or variant thereof. In some embodiments, the sequence-specific oligonucleotide comprises DNA, RNA, or a combination thereof.

In some embodiments, the solution comprises at least one selected from the group consisting of divalent cations, nucleotide triphosphates (NTPs), deoxynucleotide triphosphates (dNTPs), chemically modified NTPs, chemically modified dNTPs, chemically modified cytidine, chemically modified uridine, chemically modified guanosine, chemically modified adenosine, non-canonical NTPs, and non-canonical dNTPs. In some embodiments, the solution comprises chemically modified NTPs wherein the NTPs are chemically modified, wherein the chemical modification comprises at least one selected from the group consisting of ribose modifications, base modifications, phosphate modifications, alpha-phosphate modification, alpha-thiophosphate modifications, 3′-ribose, and 2′-ribose modifications.

In some embodiments, the primer comprises at least one selected from the group consisting of: an RNA, a chemically modified RNA, a DNA, a chemically modified DNA, a DNA-RNA chimera, a chemically modified DNA-RNA chimera, and a DNA, RNA, or DNA-RNA chimera with a non-complementary 5′-single-strand overhang. In some embodiments, the primer is complementary or partially complementary with the template, wherein a partially complementary primer has a non-complementary 5′-overhang or has a portion of bases that are not complementary with the template. In some embodiments, the primer comprises a chemically modified RNA wherein the chemical modification comprises at least one selected from the group consisting of a base modification, a ribose modification, a phosphate modification, a 2′-ribose modification, and a 3′-ribose modification. In some embodiments, the primer comprises a chemically modified DNA wherein the chemical modification comprises at least one selected from the group consisting of a base modification, a deoxyribose modification, a phosphate modification, a 2′-deoxyribose modification, and a 3′-deoxyribose modification. In some embodiments, the primer comprises a chemically modified DNA-RNA chimera, wherein the chemical modification comprises at least one selected from the group consisting of a base modification, a ribose modification, a deoxyribose modification, a phosphate modification, a 2′-ribose modification, a 2′-deoxyribose modification, a 3′-ribose modification, and a 3′-deoxyribose modification.

In a variety of embodiments, the A-family DNA polymerase mutant or variant thereof comprises a DNA polymerase theta (Polθ) mutant. In some embodiments, the DNA polymerase theta (Polθ) mutant comprises the amino acid sequence of SEQ ID NO: 1 or a variant thereof. In some embodiments, the DNA polymerase theta (Polθ) mutant comprises at least one mutation relative to SEQ ID NO:1; wherein at least one mutation is E544X or E544G; and wherein X is any proteogenic amino acid. In some embodiments, the DNA polymerase theta (Polθ) mutant further comprises at least one mutation selected from the group consisting of I535X and I535F; wherein X is any proteogenic amino acid.

In some embodiments, the DNA polymerase theta (Polθ) mutant comprises the amino acid sequence of SEQ ID NO: 17 or a variant thereof. In some embodiments, the DNA polymerase theta (Polθ) mutant comprises at least one mutation relative to SEQ ID NO:17; wherein at least one mutation is E2335X or E2335G; and wherein X is any proteogenic amino acid. In some embodiments, the DNA polymerase theta (Polθ) mutant further comprises at least one mutation selected from the group consisting of I2326X and I2326F; wherein X is any proteogenic amino acid.

In some embodiments, the DNA polymerase theta (Polθ) mutant comprises the amino acid sequence of SEQ ID NO:21 or a variant thereof.

In a variety of embodiments, the disclosure provides kits for preparing an oligonucleotide comprising a DNA polymerase theta (Polθ) mutant comprising the amino acid sequence of SEQ ID NO: 1, a variant thereof, SEQ ID NO:17, or a variant thereof. In some embodiments, the DNA polymerase theta (Polθ) mutant comprises the amino acid sequence of SEQ ID NO:1; wherein the mutant comprises at least one mutation relative to SEQ ID NO:1; wherein at least one mutation is E544X or E544G; and wherein X is any proteogenic amino acid. In some embodiments, the DNA polymerase theta (Polθ) mutant further comprises at least one mutation selected from the group consisting of I535X or I535F; wherein X is any proteogenic amino acid.

In some embodiments, the DNA polymerase theta (Polθ) mutant comprises the amino acid sequence of SEQ ID NO:17; wherein the mutant comprises at least one mutation relative to SEQ ID NO:17; wherein at least one mutation is E2335X or E2335G; and wherein X is any proteogenic amino acid. In some embodiments, the DNA polymerase theta (Polθ) mutant further comprises at least one mutation selected from the group consisting of I2326X and I2326F; wherein X is any proteogenic amino acid.

In some embodiments, the kit further comprises one or more solutions comprising one or more selected from the group consisting of: a divalent cation, NTPs, dNTPs, chemically modified NTPs, chemically modified dNTPs, Tris hydrochloride, glycerol, NP-40, bovine serum albumin (BSA), sodium chloride (NaCl), 1,4-dithiothreitol (DTT), and instructional material.

In various embodiments, the disclosure provides a DNA polymerase theta (Polθ) mutant comprising the amino acid sequence of SEQ ID NO:1; wherein the mutant comprises at least one mutation; wherein at least one mutation is E544X or E544G; and wherein X is any proteogenic amino acid. In some embodiments, the mutant further comprises a mutation selected from the group consisting of I535X and I535F; wherein X is any proteogenic amino acid. In various embodiments, the disclosure provides a DNA polymerase theta (Polθ) mutant comprising the amino acid sequence of SEQ ID NO:17; wherein the mutant comprises at least two mutations; wherein a first mutation is E2335X or E2335G; wherein a second mutation is I2326X or I2326F; and wherein each instance of X is independently any proteogenic amino acid. In various embodiments, the disclosure provides a DNA polymerase theta (Polθ) mutant comprising the amino acid sequence of SEQ ID NO:21.

In one embodiment, the disclosure provides a composition comprising a DNA polymerase theta (Polθ) mutant comprising the amino acid sequence of SEQ ID NO: 1; wherein the mutant comprises at least one mutation; wherein at least one mutation is E544X or E544G; and wherein X is any proteogenic amino acid or a nucleic acid encoding said DNA polymerase theta (Polθ) mutant. In one embodiment, the mutant further comprises a mutation selected from the group consisting of I535X and I535F; wherein X is any proteogenic amino acid. In one embodiment, the disclosure provides a composition comprising the DNA polymerase theta (Polθ) mutant comprising the amino acid sequence of SEQ ID NO:17; wherein the mutant comprises at least two mutations; wherein a first mutation is E2335X or E2335G; wherein a second mutation is I2326X or I2326F; and wherein each instance of X is independently any proteogenic amino acid. In one embodiment, the disclosure provides a composition comprising a DNA polymerase theta (Polθ) mutant comprising the amino acid sequence of SEQ ID NO:21.

### BRIEF DESCRIPTION OF THE DRAWINGS

The following detailed description of embodiments of the invention will be better understood when read in conjunction with the appended drawings. For the purpose of illustrating the invention, there are shown in the drawings embodiments which are exemplary. It should be understood, however, that the invention is not limited to the precise arrangements and instrumentalities of the embodiments shown in the drawings.

FIG. 1 depicts a representative image of the superposition of Taq DNAP and Polθ crystal structures. Superposition of Taq DNAP (blue; PDB ID: 1qss) and Polθ (green; PDB ID: 4x0q) bound to DNA/DNA templates with incoming ddGTP shows close alignment of their respective steric-gate residues.

FIG. 2 depicts representative denaturing gels showing pre-mature termination of Taq DNAP E615G on the indicated DNA/DNA primer-template in the presence of NTPs (left) and exonuclease activity by Taq DNAP E615G on the indicated RNA/DNA primer-template in the presence of NTPs (right).

FIG. 3 depicts a representative image of a superposition of Polθ: DNA/RNA and Polθ DNA/DNA crystal structures. Superposition of Polθ DNA/RNA (red; PDB ID: 6XBU) and Polo: DNA/DNA (pink; PDB ID: 4x0q) with incoming ddGTP reveals significant conformational changes in the thumb and fingers subdomain of Polθ when bound to A-form DNA/RNA.

FIG. 4 depicts a representative image of a denaturing gel showing efficient extension of RNA/DNA by WT Polθ in the presence of dNTPs.

FIG. 5 depicts a representative image of a denaturing gel showing premature termination by WT Polθ on RNA/DNA in the presence of NTPs.

FIG. 6 depicts representative images of denaturing gels showing efficient DNA-dependent RNA synthesis by PolθRP1 on a DNA/DNA primer-template (left) and an RNA/DNA primer-template (right) in the presence of NTPs, MgCl₂, and indicated NaCl titration. Percent extension for each run is depicted below its corresponding lane.

FIG. 7 depicts representative images of denaturing gels showing efficient DNA-dependent RNA synthesis by PolθRP2 on the indicated RNA/DNA primer-templates in the presence of NTPs, MgCl₂, and indicated NaCl titration. Percent extension for each run is depicted below its corresponding lane.

FIG. 8 depicts a representative image of a denaturing gel showing efficient DNA-dependent RNA synthesis by PolθRP1 on the indicated RNA/DNA primer-template in the presence of NTPs, MgCl₂, and indicated NaCl titration. Percent extension for each run is depicted below its corresponding lane.

FIG. 9 depicts a representative image of a denaturing gel showing RNaseH degradation of the RNA portion of the RNA/DNA hybrid synthesized by PolθRP2.

FIG. 10 depicts a representative image of a denaturing gel showing T7 RNAP promoter dependent synthesis of RNA with an expected length of 95 nt.

FIG. 11 depicts a representative image of a denaturing gels showing time courses of PolθRP1 and T7 RNAP on the indicated RNA/DNA primer-template in the presence of canonical NTPs.

FIG. 12 depicts representative images of denaturing gels showing time courses of PolθRP1 and T7 RNAP on the indicated RNA/DNA primer-template in the presence of canonical NTPs. T7 RNAP and PolθRP1 were pre-incubated with the RNA/DNA template for 10 min (right) or unincubated (left). Percent extension for each run is depicted below its corresponding lane.

FIG. 13 depicts representative images of denaturing gels showing RNA extension by T7 RNAP following a 10 min pre-incubation period on the indicated RNA/DNA with UTP (left) or GTP (right).

FIG. 14 depicts representative images of denaturing gels showing RNA extension by PolθRP1 following a 10 min pre-incubation period on the indicated RNA/DNA with UTP (left) or GTP (right).

FIG. 15 depicts representative images of denaturing gels demonstrating PolθRP2 time-dependent incorporation of canonical UTP and base-modified pseudouridine-triphosphate on the indicated RNA/DNA primer-template.

FIG. 16 depicts a scatter plot comparing the relative rates of PolθRP2 incorporation of UTP and pseudouridine-triphosphate from FIG. 15. Data presented as mean±S.D.; n=3.

FIG. 17 depicts representative images of denaturing gels demonstrating PolθRP2 time-dependent incorporation of the canonical CTP and base-modified 5-methyl-CTP on the indicated RNA/DNA primer-template.

FIG. 18 depicts a scatter plot comparing the relative rates of PolθRP2 incorporation of CTP and 5-methyl-CTP from FIG. 17. Data presented as mean±S.D.; n=3.

FIG. 19 depicts representative images of denaturing gels demonstrating PolθRP2 time-dependent incorporation of the canonical ATP and base-modified N6-methyl-ATP on the indicated RNA/DNA primer-template.

FIG. 20 depicts a scatter plot comparing the relative rates of PolθRP2 incorporation of ATP and N6-methyl-ATP from FIG. 19. Data presented as mean±S.D.; n=3.

FIG. 21 depicts a representative image of a denaturing gel demonstrating PolθRP2 time dependent incorporation of pseudouridine-triphosphate on the indicated RNA/DNA primer-template.

FIG. 22 depicts a schematic representation of a method for modifying the 3′ terminal end of RNA by Polθ steric-gate variants.

FIG. 23 depicts a representative image of a denaturing gel showing time courses of DNA-dependent RNA synthesis by PolθRP2 in the presence of the indicated combination of NTPs.

FIG. 24 depicts a schematic representation of RNA/DNA primer-templates.

FIG. 25 depicts a representative image of a denaturing gel showing time courses of PolθRP1 extension of RNA/DNA Template 1 of FIG. 24 in the presence of the indicated 2′-O-Me-NTPs and Mg²⁺ (left) or Mn²⁺ (right).

FIG. 26 depicts a representative image of a denaturing gel showing a time course of PolθRP1 extension of RNA/DNA Template 2 of FIG. 24 in the presence of the indicated 2′-O-Me-NTPs and Mg²⁺.

FIG. 27 depicts a representative image of a denaturing gel demonstrating Polθ steric-gate variant synthesis of phosphorothioate modified RNA. The denaturing gel shows a time course of PolθRP1 DNA-dependent RNA synthesis in the presence of all four 1′-phosphorothioate-NTP analogs with 1 mM DTT, 5 mM MgCl₂ and 1 mM MnCl₂.

FIG. 28 depicts a schematic representation of an RNA/DNA template. Black stars indicate the location of three consecutive 2′-ribose modifications at the 5′ terminus.

FIG. 29 depicts a representative image of a denaturing gel showing a time course of PolθRP1 extension of the 5′-modified RNA/DNA template in FIG. 28 in the presence of 2′-O-Me NTPs and MgCl₂.

FIG. 30 depicts a representative image of a denaturing gel showing a time course of PolθRP1 extension of the 5′-modified RNA/DNA template in FIG. 28 in the presence of 2′-MOE (2′-CH₂—O—CH₂—CH₃) NTPs and MgCl₂.

FIG. 31 depicts a representative image of a denaturing gel showing a time course of PolθRP1 extension of the 5′-modified RNA/DNA template in FIG. 28 in the presence of 2′-F NTPs and MgCl₂.

FIG. 32 depicts a representative image of a denaturing gel showing a time course of PolθRP1 extension of the 5′-modified RNA/DNA template in FIG. 28 in the presence 2′-H NTPs and MgCl₂.

FIG. 33 depicts a representative image of a denaturing gel showing a time course of PolθRP1 extension of the indicated 5′-Cy3-modified RNA/DNA template depicted in the presence of NTPs and MgCl₂.

FIG. 34 depicts a schematic representation of the preparation of cDNA for sequencing.

FIG. 35 depicts a representative bar plot showing the number of variations versus number of reads.

FIG. 36 depicts a table showing the percent of reads with the indicated number of variations of FIG. 35 from the reference sequence.

FIG. 37 depicts a representative image of a denaturing gel showing PolθRP1 extension of the indicated RNA/DNA in the presence of the indicated combination of NTPs.

FIG. 38 depicts a representative image of a denaturing gel showing PolθRP1 extension of the indicated RNA/DNA in the presence of the indicated combination of NTPs.

FIG. 39 depicts a representative image of a denaturing gel showing PolθRP1 extension of the indicated RNA/DNA in the presence of the indicated combination of NTPs.

FIG. 40 depicts representative images of denaturing gels showing PolθRP1 extension of the indicated RNA/DNA primer-template in the presence of the UTP and ATP (left) or wTP and ATP (right) at the concentrations indicated. Percent misincorporation of AMP in each run is indicated below its corresponding lane.

FIG. 41 depicts representative images of denaturing gels showing PolθRP1 extension of the indicated RNA/DNA primer-template in the presence of the CTP and ATP (left) or 5-methyl-CTP and ATP (right) at the concentrations indicated. Percent misincorporation of AMP in each run is indicated below its corresponding lane.

FIG. 42 depicts a representative image of a denaturing gel showing DNA-dependent RNA synthesis by a PolθDL steric gate mutant protein in the presence of canonical NTPs (lane 2) and 2′-O-methyl NTPs (lane 3).

### DETAILED DESCRIPTION

The present invention is based on the discovery that human DNA polymerase theta (Polθ) variants can be utilized to synthesize long (95-200 nt) sequence-specific RNA oligonucleotides with canonical ribonucleotides and ribonucleotide analogs commonly used for stabilizing RNA for therapeutic and genome engineering applications. In contrast to natural promoter-dependent RNA polymerases, Polθ variants synthesize RNA by initiating from DNA or RNA primers which enables the template-dependent production of highly pure sequence-specific RNA products. Remarkably, Polθ variants show lower capacity to incorporate incorrect ribonucleotides compared to T7 RNA polymerase.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

“Amplification” refers to any means by which a polynucleotide sequence is copied and thus expanded into a larger number of polynucleotide molecules, e.g., by reverse transcription, polymerase chain reaction, and ligase chain reaction, among others. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 5%, or at least about 6%, or at least about 7%, or at least about 8%, or at least about 9%, or at least about 10%, or at least about 20%, or at least about 30%, or at least about 40%, or at least about 50%, or at least about 75%, or at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “fragment,” as applied to a nucleic acid or protein, refers to a subsequence of a larger nucleic acid or protein, respectively. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between). A “fragment” of a protein can be at least about 15 amino acids in length; for example, at least about 50 amino acids to about 100 amino acids; at least about 100 to about 500 amino acids, at least about 500 to about 1000 amino acids, at least about 1000 amino acids to about 1500 amino acids; or about 1500 amino acids to about 2500 amino acids; or about 2500 amino acids (and any integer value in between).

“Homologous, homology” or “identical, identity” as used herein, refer to comparisons among amino acid and nucleic acid sequences. When referring to nucleic acid molecules, “homology,” “identity,” or “percent identical” refers to the percent of the nucleotides of the subject nucleic acid sequence that have been matched to identical nucleotides by a sequence analysis program. Homology can be readily calculated by known methods. Nucleic acid sequences and amino acid sequences can be compared using computer programs that align the similar sequences of the nucleic or amino acids and thus define the differences. In some methodologies, the BLAST programs (NCBI) and parameters used therein are employed, and the ExPaSy is used to align sequence fragments of genomic DNA sequences. However, equivalent alignment assessments can be obtained through the use of any standard alignment software.

As used herein, “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′ATTGCC 3′ and 5′TATGGC 3′ share 50% homology.

“Hybridization probes” are oligonucleotides capable of binding in a base-specific manner to a complementary strand of nucleic acid. Such probes include peptide nucleic acids, as described in Nielsen et al., 1991, Science 254, 1497-1500, and other nucleic acid analogs and nucleic acid mimetics. See U.S. Pat. No. 6,156,501.

The term “hybridization” refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example, two complementary or partially complementary sequences. The hybrid may have double-stranded regions and single-stranded regions. The hybrid may be, for example, DNA: DNA, RNA: DNA or DNA: RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

The stability of a hybrid depends on a variety of factors including the length of complementarity, the presence of mismatches within the complementary region, the temperature, and the concentration of salt in the reaction. Hybridizations are usually performed under stringent conditions, for example, at a salt concentration of no more than 1 M and a temperature of at least 25° C. For example, conditions of 5×SSPE (750 mM NaCl, 50 mM Na Phosphate, 5 mM EDTA, pH 7.4) or 100 mM MES, 1 M Na, 20 mM EDTA, 0.01% Tween-20 and a temperature of 25-50° C. are suitable for allele-specific probe hybridizations. In some embodiments, hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual available from Affymetrix (Santa Clara, CA).

A first oligonucleotide anneals with a second oligonucleotide with “high stringency” if the two oligonucleotides anneal under conditions whereby only oligonucleotides which are at least about 75%, or at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g., Sambrook et al., 2012, Molecular Cloning: A Laboratory Manual, Cold Spring Harbor Laboratory Press, Cold Spring Harbor, N.Y.).

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient. As used herein, “isolate” refers to a nucleic acid obtained from an individual, or from a sample obtained from an individual. The nucleic acid may be analyzed at any time after it is obtained (e.g., before or after laboratory culture, before or after amplification.)

As used herein, the term “purified” or “to purify” refers to the removal of components (e.g., contaminants) from a sample. For example, nucleic acids are purified by removal of contaminating cellular proteins or other undesired nucleic acid species. The removal of contaminants results in an increase in the percentage of desired nucleic acid in the sample.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The term “mismatch,” “mismatch control” or “mismatch probe” refers to a nucleic acid whose sequence is not perfectly complementary to a particular target sequence. The mismatch may comprise one or more bases. While the mismatch(es) may be located anywhere in the mismatch probe, terminal mismatches are less desirable because a terminal mismatch is less likely to prevent hybridization of the target sequence. In some embodiments, the mismatch is located at or near the center of the probe such that the mismatch is most likely to destabilize the duplex with the target sequence under the test hybridization conditions.

As used herein, the term “nucleic acid” refers to both naturally occurring molecules such as DNA and RNA, but also various derivatives and analogs. Generally, the probes, hairpin linkers, and target polynucleotides of the present teachings are nucleic acids, and typically comprise DNA. Additional derivatives and analogs can be employed as will be appreciated by one having ordinary skill in the art.

The term “nucleotide base,” as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6 delta 2-isopentenyladenine (6iA), N6-delta 2-isopentenyl-2-methylthioadenine (2 ms6iA), N2-dimethylguanine (dmG), 7methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, 06-methylguanine, N6-methyladenine, 04-methylthymine, 5,6-dihydrothymine, 5,6-dihydrouracil, 5-methylcytosine, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C₁-C₆)alkoxyribose, 2′-(C₅-C₁₄) aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C₁-C₆)alkylribose, 2′-deoxy-3′-(C₁-C₆)alkoxyribose and 2′-deoxy-3′-(C₅-C₁₄) aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-anomeric nucleotides, 1′-anomeric nucleotides, 2′-4′- and 3′-4′-linked and other “locked” or “LNA,” bicyclic sugar modifications (see, e.g., PCT published application nos. WO 98/22489, WO 98/39352; and WO 99/14226). The term “nucleic acid” typically refers to large polynucleotides.

The term “nucleotide analogs” as used herein refers to modified or non-naturally occurring nucleotides including, but not limited to, analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242; B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having one or more modification son the phosphate moiety, base moiety, or sugar moiety, such as dideoxy nucleotides and 2′-O-methyl nucleotides. Nucleotide analogs include modified forms of deoxyribonucleotides as well as ribonucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning and amplification technology, and the like, and by synthetic means. An “oligonucleotide” as used herein refers to a short polynucleotide, typically less than 100 bases in length.

Conventional notation is used herein to describe polynucleotide sequences: the left-hand end of a single-stranded polynucleotide sequence is the 5′-end. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.” In the sequences described herein:
A=adenine,
G-guanine,
T=thymine,
C=cytosine,
U=uracil,
H=A, C or T/U,
R=A or G,
M=A or C,
K=G or T/U,
S=G or C,
Y=C or T/U,
W=A or T/U,
B=G or C or T/U,
D=A or G, or T/U,
V=A or G or C,
N=A or G or C or T/U.

The skilled artisan will understand that all nucleic acid sequences set forth herein throughout in their forward orientation, are also useful in the compositions and methods of the invention in their reverse orientation, as well as in their forward and reverse complementary orientation, and are described herein as well as if they were explicitly set forth herein.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, e.g., in the presence of nucleotides, a complementary polynucleotide template, and an agent for polymerization such as DNA polymerase. A primer is typically single-stranded but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis but need not reflect the exact sequence of the template. In such a case, specific hybridization of the primer to the template depends on the stringency of the hybridization conditions. Primers can be labeled with a detectable label, e.g., chromogenic, radioactive, or fluorescent moieties and used as detectable moieties. Examples of fluorescent moieties include, but are not limited to, rare earth chelates (europium chelates), Texas Red, rhodamine, fluorescein, dansyl, phycocrytherin, phycocyanin, spectrum orange, spectrum green, and/or derivatives thereof. Other detectable moieties include digoxigenin and biotin.

As used herein a “probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, a linkage other than a phosphodiester bond may join the bases in probes, so long as it does not interfere with hybridization. Thus, probes may be peptide nucleic acids in which the constituent bases are joined by peptide bonds rather than phosphodiester linkages. The term “match,” “perfect match,” “perfect match probe” or “perfect match control” refers to a nucleic acid that has a sequence that is perfectly complementary to a particular target sequence. The nucleic acid is typically perfectly complementary to a portion (subsequence) of the target sequence. A perfect match (PM) probe can be a “test probe,” a “normalization control” probe, an expression level control probe, and the like. A perfect match control or perfect match is, however, distinguished from a “mismatch” or “mismatch probe.”

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, oligonucleotides and nucleic acids.

“Variant” as the term is used herein, is a nucleic acid sequence or a peptide sequence that differs in sequence from a reference nucleic acid sequence or peptide sequence respectively but retains essential properties of the reference molecule. Changes in the sequence of a nucleic acid variant may not alter the amino acid sequence of a peptide encoded by the reference nucleic acid, or may result in amino acid substitutions, additions, deletions, fusions, and truncations. A variant of a nucleic acid or peptide can be a naturally occurring such as an allelic variant or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention provides compositions and method for preparing long (>95 nt) sequence-specific oligonucleotides. In some embodiments, the oligonucleotides are DNA, RNA, or a combination of the two. In some embodiments, the template-dependent RNA synthesis is performed by an A-family DNA polymerase or variant thereof. In one embodiment, the A-family DNA polymerase is a Polθ or a variant thereof. In one embodiment, the Polθ possesses robust DNA template-dependent RNA polymerase activity in the absence of a promoter and exclusively in the presence of a divalent cation.

In some embodiments, the invention provides methods of preparing long sequence-specific RNA oligonucleotides. In one embodiment, the method incorporates canonical ribonucleotides into the long sequence-specific RNA oligonucleotide. In some embodiments, the modified ribonucleotides comprise non-canonical ribonucleotides. In some embodiments, the method incorporates modified ribonucleotides and deoxyribonucleotides into the long RNA oligonucleotide.

### Compositions

In one embodiment, the invention provides recombinant Polθ In some aspects, the invention includes an isolated protein (e.g. Polθ), wherein the protein is used to synthesize an RNA oligonucleotide. In some embodiments, the isolated protein is an A-family polymerase. In some embodiments, the A-family polymerase is a eukaryotic A-family polymerase. In some embodiments, the eukaryotic A-family polymerase is an A-family polymerase from one selected from the group consisting of: an invertebrate, a vertebrate, a human, and a plant.

In various embodiments, the invention provides compositions comprising an A-family polymerase. In some embodiments, the A-family is a DNA polymerase theta (Polθ). In some embodiments, the Polθ is a human Polθ or a variant or mutant thereof. In one embodiment, the Polθ comprises an amino acid sequence of SEQ ID NO:17 or a variant or mutant thereof. In some embodiments, the Polθ mutant comprises at least an E2335X mutation relative to SEQ ID NO:17, wherein X is any proteogenic amino acid. Proteogenic amino acids are any one of those selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), pyrrolysine (O), proline (P), glutamine (Q), arginine (R), serine(S), threonine (T), selenocysteine (U), valine (V), tryptophan (W), and tyrosine (Y). In one embodiment, the mutation is an E2245G mutation (PolθRP1, SEQ ID NO:18).

In some embodiments, the Polθ comprises at least both an E2335X and I2326X mutation relative to SEQ ID NO:17, wherein each instance of X is independently any proteogenic amino acid. In some embodiments, the Polθ comprises an E2335G mutation and an I2326X mutation, wherein X is any proteogenic amino acid. In some embodiments, the Polθ comprises an I2326F mutation and an E2335X mutation, wherein X is any proteogenic amino acid. In some embodiments, the Polθ comprises an E2335G mutation and an I2326F mutation (PolθRP2, SEQ ID NO:19). In some embodiments, the Polθ comprises an E2335G mutation and various other mutations.

In yet another embodiment, the Polθ is a functional fragment of full-length Polθ or a variant or mutant thereof. In certain embodiments, the Polθ is a functional fragment of human Polθ Examples of functional fragments of Polθ include, but are not limited to, fragments comprising an amino acid sequence of SEQ ID NO: 17 from about amino acid 1, about amino acid 2, about amino acid 3, about amino acid 4, about amino acid 5, about amino acid 6, about amino acid 7, about amino acid 8, about amino acid 9, about amino acid 10, about amino acid 15, about amino acid 10, about amino acid 15, about amino acid 20, about amino acid 25, about amino acid 30, about amino acid 35, about amino acid 40, about amino acid 45, about amino acid 50, about amino acid 60, about amino acid 70, about amino acid 80, about amino acid 90, about amino acid 100, about amino acid 120, about amino acid 140, about amino acid 160, about amino acid 180, about amino acid 200, about amino acid 250, about amino acid 300, about amino acid 350, about amino acid 400, about amino acid 450, about amino acid 500, about amino acid 550, about amino acid 600, about amino acid 650, about amino acid 700, about amino acid 750, about amino acid 800, about amino acid 850, about amino acid 900, about amino acid 950, about amino acid 1000, about amino acid 1050, about amino acid 1100, about amino acid 1150, about amino acid 1200, about amino acid 1250, about amino acid 1300, about amino acid 1350, about amino acid 1400, about amino acid 1450, about amino acid 1500, about amino acid 1550, about amino acid 1600, about amino acid 1650, about amino acid 1700, about amino acid 1750, about amino acid 1790, about amino acid 1792, about amino acid 1794, about amino acid 1800, about amino acid 1817, about amino acid 1819, about amino acid 1821, about amino acid 1850, or about amino acid 1900 through about amino acid 2400, about amino acid 2450, about amino acid 2500, about amino acid 2510, about amino acid 2520, about amino acid 2530, about amino acid 2540, about amino acid 2545, about amino acid 2550, about amino acid 2555, about amino acid 2560, about amino acid 2565, about amino acid 2570, about amino acid 2575, about amino acid 2580, about amino acid 2581, about amino acid 2582, about amino acid 2583, about amino acid 2584, about amino acid 2585, about amino acid 2586, about amino acid 2587, about amino acid 2588, about amino acid 2589, or about amino acid 2590. In some embodiments, the functional fragment of Polθ comprises at least one mutation in the glutamic acid corresponding to E2335 of full Polθ (SEQ ID NO:17).

In some embodiments, the Polθ is a Polθ₁₇₉₂₋₂₅₉₀ having the amino acid sequence of SEQ ID NO: 1 or a variant or mutant thereof. In one embodiment, the Polθ comprises an amino acid sequence of SEQ ID NO: 1 or a variant or mutant thereof. In some embodiments, the Polθ further comprises at least an E544X mutation relative to SEQ ID NO:1, wherein X is any proteogenic amino acid. Proteogenic amino acids are any one of those selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), pyrrolysine (O), proline (P), glutamine (Q), arginine (R), serine(S), threonine (T), selenocysteine (U), valine (V), tryptophan (W), and tyrosine (Y). In one embodiment, the mutation is an E544G mutation.

In some embodiments, the Polθ comprises at least both an E544X and I535X mutation relative to SEQ ID NO:1, wherein each instance of X is independently any proteogenic amino acid. In some embodiments, the Polθ comprises an E544G mutation and an I535X mutation, wherein X is any proteogenic amino acid. In some embodiments, the Polθ comprises an I535F mutation and an E544X mutation, wherein X is any proteogenic amino acid. In some embodiments, the Polθ comprises an E544G mutation and an I535F mutation.

In some embodiments, the functional fragment of Polθ is a Polθ₁₈₁₉₋₂₅₉₀ having the amino acid sequence of SEQ ID NO:20 or a variant or mutant thereof. In one embodiment, the Polθ comprises an amino acid sequence of SEQ ID NO:20 or a variant or mutant thereof. In some embodiments, the Polθ further comprises at least an E517X mutation relative to SEQ ID NO:20, wherein X is any proteogenic amino acid. Proteogenic amino acids are any one of those selected from the group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), pyrrolysine (O), proline (P), glutamine (Q), arginine (R), serine(S), threonine (T), selenocysteine (U), valine (V), tryptophan (W), and tyrosine (Y). In one embodiment, the mutation is an E517G mutation.

In some embodiments, the Polθ comprises at least both an E517X and I508X mutation relative to SEQ ID NO:20, wherein each instance of X is independently any proteogenic amino acid. In some embodiments, the Polθ comprises an E517G mutation and an I508X mutation, wherein X is any proteogenic amino acid. In some embodiments, the Polθ comprises an 1508F mutation and an E517X mutation, wherein X is any proteogenic amino acid. In some embodiments, the Polθ comprises an E517G mutation and an I508F mutation.

In some embodiments, the functional fragment of Polθ is a PolθDL having the amino acid sequence of SEQ ID NO:21 or a variant or mutant thereof.

In one embodiment, the invention is a recombinant Polθ Thus, the invention encompasses compositions and methods for producing recombinant Polθ including but is not limited to, expression vectors, methods for the introduction of exogenous DNA into cells with concomitant expression of the exogenous DNA in the cells, and methods of protein modification, expression and isolation, such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). In some embodiments Polθ is encoded by the human POLQ gene. In some embodiments Polθ is encoded by the C. elegans polq-1 gene. In some embodiments Polθ is encoded by the mouse Polq gene.

In some embodiments, the protein is a fragment or mutant of a protein which is able to synthesize an RNA oligonucleotide comprised of a specific sequence. Therefore, another embodiment of the invention is to provide an isolated nucleic acid molecule that encodes the protein fragment or the mutated or variant protein. According to the invention, the protein fragment or mutated protein is obtained by mutating the wildtype protein coding sequence. The mutagenesis technique could be by chemical, error prone PCR or site-directed approach. The suitable technique can be selected and used for introducing mutations and the mutated nucleic acid molecule can be cloned and expressed and the activity of the protein can be determined.

The isolated nucleic acid sequence encoding the protein fragment or mutated protein can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding the mutated protein, or functional fragment thereof. In one embodiment, the composition comprises an isolated RNA molecule encoding the mutated protein, or a functional fragment thereof.

The desired polynucleotide can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector, and a mammalian cell vector. Numerous expression vector systems exist that comprise at least a part or all of the compositions described elsewhere herein. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the desired polynucleotide, at least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

One such promoter sequence is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In the context of an expression vector, the vector can be readily introduced into a host cell, e.g., mammalian, bacterial, yeast or insect cell by any method in the art. For example, the expression vector can be transferred into a host cell by physical, chemical, or biological means. It is readily understood that the introduction of the expression vector comprising the polynucleotide of the invention yields a silenced cell with respect to a regulator.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. One colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Any DNA vector or delivery vehicle can be utilized to transfer the desired polynucleotide to a cell in vitro or in vivo. In the case where a non-viral delivery system is utilized, one suitable delivery vehicle is a liposome. Some of the delivery systems and protocols described elsewhere can be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine nucleic acid complexes.

Transformation refers to the transfer of a nucleic acid (e.g., exogenous nucleic acid) into the genome of a host microorganism, resulting in genetically stable inheritance. Host microorganisms containing the transformed nucleic acid are referred to as “non-naturally occurring” or “recombinant” or “transformed” or “transgenic” microorganisms. Host microorganisms may be selected from, and the non-naturally occurring microorganisms generated in, any prokaryotic or eukaryotic microbial species from the domains of Archaea, Bacteria, or Eukarya. Exemplary bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungal species include Saccharomyces cerevisiae, Schizosaccharomyces pombe, Kluyveromyces lactis, Kluyveromyces marxianus, Aspergillus terreus, Aspergillus niger, Rhizopus arrhizus, Rhizopus oryzae, Candida, Yarrowia, Hansenula, Pichia pastoris, Torulopsis, Rhodotorula and Yarrowia lipolytica. It is understood that any suitable host microorganism can be used to introduce suitable genetic modifications (e.g., an exogenous nucleic acid encoding an enzyme with methane monooxygenase activity that is stable in the presence of a chemical or environmental stress) to produce a non-naturally occurring microorganism as provided in the specification.

Reference proteins or nucleic acids, also known as “wild type” or “parent” proteins and nucleic acids are used as starting molecules for genetic engineering of variant enzymes with the desired stability.

Expression of recombinant proteins is often difficult outside their original host. For example, variation in codon usage bias has been observed across different species of bacteria (Sharp et al., 2005, Nucl. Acids. Res. 33:1141-1153). Over-expression of recombinant proteins even within their native host may also be difficult. In certain embodiments of the invention, nucleic acids (e.g., a nucleic acid encoding an enzyme with activity that is stable in the presence of a chemical or environmental stress) that are to be introduced into microorganisms according to any of the embodiments disclosed herein may undergo codon optimization to enhance protein expression. Codon optimization refers to alteration of codons in genes or coding regions of nucleic acids for transformation of an organism to reflect the typical codon usage of the host organism without altering the polypeptide for which the DNA encodes. Codon optimization methods for optimum gene expression in heterologous organisms are known in the art and have been previously described (see., e.g., Welch et al., 2009, PLOS One 4: e7002; Gustafsson et al., 2004, Trends Biotechnol. 22:346-353; Wu et al., 2007, Nucl. Acids Res. 35: D76-79; Villalobos et al., 2006, BMC Bioinformatics 7:285; U.S. Patent Publication 2011/0111413; and U.S. Patent Publication 2008/0292918).

The protein of the present invention may be made using chemical methods. For example, peptides can be synthesized by solid phase techniques (Roberge J Y et al (1995) Science 269:202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide.

The Escherichia coli (E. coli) Rosetta2 (DE3)/pLysS strains, or other E. coli strains, may be transformed with a vector to express the modified protein and expressed in a flask or fermenter. The cells may be grown in the autoinduction medium (1× Terrific Broth, 0.5% w/v glycerol, 0.05% w/v dextrose, 0.2% w/v alpha-lactose, 100 μg/ml ampicillin and 34 μg/ml chloramphenicol) and cells harvested anytime between 60 to 70 hours after inoculation.

The cell pellet obtained by fermentation or centrifugation may be lysed after addition of suitable amount of resuspension buffer, by any method not limited to sonication, high pressure homogenization, bead mill, freeze thawing or by addition of any chemical.

According to the invention, the enzyme produced by fermentation may be enriched to obtain enzyme as usable for the activity, by one or combination of methods. Methods of protein purification are known in the art. See, for example, Sambrook et al. (2012, Molecular Cloning: A Laboratory Manual, Cold Spring Harbor Laboratory, New York). The methods may involve binding of the protein to any matrix with diethylaminoethyl (DEAE) or other weak anion exchange functional group in the presence of Tris buffer or phosphate buffer and eluting with 0.4M sodium chloride (NaCl) solution. Alternatively, the methods may involve binding of the protein to matrix with Ni²⁺ or other divalent metal cation affinity functional group in the presence of Tris buffer or phosphate buffer, or other buffer, and eluting with 0.5 M or other concentrations of Imidazole solution. An alternate method may involve addition of 0.3% (v/w) of polyethyleneimine (PEI) to cell lysate and trapping the enzyme in the formed pellet or releasing the enzyme from the pellet into solution with the addition of 0.4M NaCl. In yet another alternate method 0.1% (v/v) PEI may be added stirred for suitable time, about 1 hour, and centrifuged. To the centrifugate 60% ammonium sulfate (w/w) may be added, followed by stirring over a period of time and centrifuged. The pellet obtained with the active protein may be used for further processing. The active protein thus obtained from any of the processes described herein may be used as solution or as lyophilized solid or as an immobilized solid or as a granule.

The composition of a protein may be confirmed by amino acid analysis or sequencing.

The variants of the proteins according to the present invention may be (i) one in which one or more of the amino acid residues are substituted with a conserved or non-conserved amino acid residue (such as a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, such as different from the original sequence in less than 40% of residues per segment of interest, or different from the original sequence in less than 25% of residues per segment of interest, or different by less than 10% of residues per segment of interest, or different from the original protein sequence in just a few residues per segment of interest and at the same time sufficiently homologous to the original sequence to preserve the functionality of the original sequence and/or the ability to stimulate the differentiation of a stem cell into the osteoblast lineage. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences can be determined by using the BLASTP algorithm [BLAST Manual, Altschul, S., et al., NCBI NLM NIH Bethesda, Md. 20894, Altschul, S., et al., J. Mol. Biol. 215:403-410 (1990)].

The proteins of the invention can be post-translationally modified. For example, post-translational modifications that fall within the scope of the present invention include signal peptide cleavage, glycosylation, acetylation, isoprenylation, proteolysis, myristoylation, parylation, ubiquitylation, sumoylation, phosphorylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The proteins of the invention may include unnatural amino acids formed by post-translational modification or by introducing unnatural amino acids during translation. A variety of approaches are available for introducing unnatural amino acids during protein translation.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains its intended functionality.

A peptide or protein of the invention may be phosphorylated using conventional methods such as the method described in Reedijk et al. (The EMBO Journal 11 (4): 1365, 1992).

### Nucleic Acids

In one aspect, the invention provides nucleic acids which can be used as a template for RNA synthesis by Polθ In another aspect, the invention provides nucleic acid which can be extended by Polθ The nucleic acids, as well as the substrate of the invention, may be from any source. Nucleic acid in the context of the present invention includes but is not limited to deoxyribonucleic acid (DNA), ribonucleic acid (RNA), and peptide nucleic acid (PNA). DNA and RNA are naturally occurring in organisms, however, they may also exist outside living organisms or may be added to organisms. The nucleic acid may be of any origin, e.g., viral, bacterial, archae-bacterial, fungal, ribosomal, eukaryotic, or prokaryotic. It may be nucleic acid from any biological sample and any organism, tissue, cell, or sub-cellular compartment. It may be nucleic acid from any organism. The nucleic acid may be pre-treated before quantification, e.g., by isolation, purification, or modification. Also artificial or synthetic nucleic acid may be used. The length of the nucleic acids may vary. The nucleic acids may be modified, e.g. may comprise one or more modified nucleobases or modified sugar moieties (e.g., comprising methoxy groups). The backbone of the nucleic acid may comprise one or more peptide bonds as in peptide nucleic acid (PNA). The nucleic acid may comprise a base analog such as non-purine or non-pyrimidine analog or nucleotide analog. It may also comprise additional attachments such as proteins, peptides and/or or amino acids.

In one embodiment, the nucleic acid comprises single-stranded DNA (ssDNA), double stranded DNA (dsDNA), partial ssDNA (pssDNA), DNA/RNA hybrid, and telomeric ssDNA. In one embodiment, the substrate is transferred to the 3′-end of the ssDNA, pssDNA, RNA, telomeric ssDNA, or dsDNA.

In one embodiment, the substrate comprises GTP, CTP, ATP, UTP, dGTP, dCTP, dATP, dUTP, or a nucleotide analog. In some embodiments, a nucleotide or nucleotide analog can be labeled. Examples of possible labels include, but are not limited to a radioisotope, an enzyme, an enzyme cofactor, an enzyme substrate, an enzyme inhibitor, a dye, a hapten, a chemiluminescent molecule, a fluorescent molecule, a phosphorescent molecule, an electrochemiluminescent molecule, a chromophore, a magnetic particle, an affinity label, a chromogenic agent, an azide group, or other groups used for click chemistry, and other moieties known in the art.

In one embodiment, the substrate comprises a deoxyribonucleotide or ribonucleotide modified at one or more positions within the sugar moiety, tri-phosphate moiety, or base moiety. In one embodiment, the deoxyribonucleotide or ribonucleotide ribose sugar is modified. In one embodiment, the modification in the ribose moiety is a 2′-ribose modification, a 3′-ribose modification, or a combination thereof. In one embodiment, the deoxyribonucleotide or ribonucleotide tri-phosphate moiety is modified. In some embodiments, the modification to the tri-phosphate an alpha-phosphate modification or an alpha-thiophosphate modification. In one embodiment the deoxyribonucleotide or ribonucleotide base moiety is modified.

### Methods

In one aspect, the invention provides methods of generating long sequence-specific RNA oligonucleotides. The method comprises: providing an A-family member polymerase and nucleotide triphosphates (NTPs); forming a mixture comprising the A-family member polymerase, the NTPs, a template nucleic acid, a primer nucleic acid, and a reaction solution wherein the reaction mixture comprises at least one divalent metal; incubating the mixture; and isolating the RNA oligonucleotides.

In some embodiments, the template nucleic acid is a single-stranded DNA, and the mixture further comprises a complementary or partially complementary RNA or DNA strand annealed to the DNA template as a primer. Examples of partially complementary RNA or DNA primers are about at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 50%, at least about 60%, at least about 70%, at least about 80%, at least about 90%, at least about 95%, at least about 96%, at least about 97%, at least about 98%, or at least about 99% complementary.

In one embodiment, the divalent metal is magnesium (Mg²⁺), manganese (Mn²⁺), or cobalt (Co²⁺). In certain embodiments, the divalent metal is Mn²⁺. In certain embodiments, the divalent metal is Mg²⁺. In some embodiments the concentration of the divalent metal in the reaction solution is 1-50 mM. In some embodiments the concentration of the divalent metal in the reaction solution is 5-20 mM. In some embodiments the concentration of the divalent metal in the reaction solution is 5-10 mM. In certain embodiments, a mixture of divalent metals including, but not limited to, Mn²⁺ and magnesium Mg²⁺ are used.

In one embodiment, the reaction solution further comprises a buffer. In certain embodiments, the buffer is Tris HCl. In some embodiments, the pH of the buffer is between about 7.0 and about 8.2. In various embodiments, the pH of the buffer is about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, or about 8.8.

In one embodiment, the reaction solution further comprises glycerol. In some embodiments the concentration of glycerol in the reaction solution is less than 20%, In some embodiments the concentration of glycerol in the reaction solution is less than 10%.

In one embodiment, the reaction solution further comprises a non-ionic detergent. In certain embodiments, the non-ionic detergent is NP-40. In some embodiments the concentration of NP-40 in the reaction solution is less than 1%. In some embodiments the concentration of NP-40 in the reaction solution is less than 0.1%. In some embodiments the concentration of NP-40 in the reaction solution is 0.01% or less than 0.01%.

In one embodiment, the reaction solution further comprises bovine serum albumin (BSA). In some embodiments the concentration of BSA in the reaction solution is 0.1 mg/ml.

In some embodiments, the step incubating the mixture comprises incubating the mixture at a controlled temperature for a controlled length of time. In certain embodiments, the incubation temperature is 25° C.-42° C. In some embodiments, the temperature is 25° C. or 37° C. In some embodiments, the incubation time is between about 30 seconds and about 6 hours. In some embodiments, the incubation time is about 2 hours. In certain embodiments, the incubation time is less than about 2 hours. In some embodiments, the incubation time is about 15 seconds, about 30 seconds, about 60 second, about 90 seconds, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 40 minutes, about 45 minutes, about 50 minutes, about 55 minutes, or about 60 minutes.

In some embodiments, the ratio of A-family polymerase to nucleic acid is defined. In certain embodiments, the molar ratio of Polθ nucleic acid is between about 20:1 and about 1:20. In some embodiments, the molar ratio is about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 16:, about 1:7, about 1:8, about 1:9, about 1:10, about 1:11, about 1:12, about 1:13, about 1:14, about 1:15, about 1:16, about 1:17, about 1:18, about 1:19, or about 1:20.

The RNA oligonucleotide product can be isolated or amplified using a primer that corresponds to a primer binding site present in the ligated product (i.e., primer binding site present in the donor molecule or the resulting hybrid product).

In various embodiments of the invention the quantifying steps comprise a method having one or more steps selected from the group consisting of gel electrophoresis, capillary electrophoresis, labelling reactions with subsequent detection measures and quantitative real-time PCR or isothermal target amplification.

In another aspect, the invention provides methods of generating extending a nucleic acid primer-templates. In some embodiments, the method comprises one or more steps including: providing an A-family member polymerase and NTPs or deoxyNTPs (dNTPs); forming a mixture comprising the A-family member polymerase, the NTPs/dNTPs, a nucleic acid primer-template, and a reaction solution wherein the reaction mixture comprises at least one divalent metal; incubating the mixture; and isolating the extended nucleic acid.

In some embodiments, the nucleic acid primer-template is a DNA/DNA primer-template or an RNA/DNA primer-template. In some embodiments, the primer-template is a DNA/DNA primer-template. In one embodiment, the DNA/DNA primer-template has 5′-single strand DNA overhangs. In some embodiments, the primer-template is an RNA/DNA primer-template. In some embodiments, the RNA/DNA has a 5′-single-stranded DNA overhang. In some embodiments, the RNA acts as a primer for RNA synthesis.

In some embodiments, the RNA is a chemically modified RNA. In some embodiments, the RNA modification is one or more selected from the group consisting of a base modification, a ribose modification, a phosphate modification, a 2′-ribose modification, and a 3′-ribose modification.

In one embodiment, the divalent metal is magnesium (Mg²⁺), manganese (Mn²⁺), or cobalt (Co²⁺). In certain embodiments, the divalent metal is Mn²⁺. In certain embodiments, the divalent metal is Mg²⁺. In some embodiments the concentration of the divalent metal in the reaction solution is 1-50 mM. In some embodiments the concentration of the divalent metal in the reaction solution is 5-20 mM. In some embodiments the concentration of the divalent metal in the reaction solution is 5-10 mM. In certain embodiments, a mixture of divalent metals including, but not limited to, Mn²⁺ and magnesium Mg²⁺ is used.

In one embodiment, the reaction solution further comprises a buffer. In certain embodiments, the buffer is Tris HCl. In some embodiments the pH of the buffer is between about 6.5 and about 8.8. In some embodiments, the pH of the buffer is between about 7.0 and about 8.2. In various embodiments, the pH of the buffer is about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, or about 8.8.

In one embodiment, the reaction solution further comprises glycerol. In some embodiments, the concentration of glycerol in the reaction solution is less than 20. In some embodiments, the concentration of glycerol in the reaction solution is 10% or less than 10%.

In one embodiment, the reaction solution further comprises a non-ionic detergent. In certain embodiments, the non-ionic detergent is NP-40. In some embodiments the concentration of NP-40 in the reaction solution is less than 1. In some embodiments the concentration of NP-40 in the reaction solution is less than 0.1%. In some embodiments the concentration of NP-40 in the reaction solution is 0.01% or less than 0.01%.

In one embodiment, the reaction solution further comprises bovine serum albumin (BSA). In some embodiments the concentration of BSA in the reaction solution is 0.1 mg/ml.

In some embodiments, the step incubating the mixture comprises incubating the mixture at a controlled temperature for a controlled length of time. In certain embodiments, the incubation temperature is 25° C.-42° C. In some embodiments, the temperature is 25° C. or 37° C. In some embodiments, the incubation time is between about 30 seconds and about 6 hours. In some embodiments, the incubation time is about 2 hours. In certain embodiments, the incubation time is less than about 2 hours. In some embodiments, the incubation time is about 1.5 minutes, about 2 minutes, about 3 minutes, about 4 minutes, about 5 minutes, about 8 minutes, about 10 minutes, about 15 minutes, about 16 minutes, about 20 minutes, about 40 minutes, about 45 minutes, or about 60 minutes.

In some embodiments, the ratio of A-family polymerase to nucleic acid is defined. In certain embodiments, the molar ratio of Polθ nucleic acid is between about 20:1 and about 1:20. In some embodiments, the molar ratio is about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 16:, about 1:7, about 1:8, about 1:9, about 1:10, about 1:11, about 1:12, about 1:13, about 1:14, about 1:15, about 1:16, about 1:17, about 1:18, about 1:19, or about 1:20.

The RNA oligonucleotide product can be isolated or amplified using a primer that corresponds to a primer binding site present in the ligated product (i.e., primer binding site present in the donor molecule or the resulting hybrid product).

In particular embodiments of the invention the quantifying steps comprise a method selected from the group consisting of gel electrophoresis, capillary electrophoresis, labelling reactions with subsequent detection measures and quantitative real-time PCR or isothermal target amplification.

Thus, in one embodiment, the invention provides a method of synthesizing sequence-specific RNA, comprising the steps of: providing a DNA template in a solution comprising divalent cations and nucleotide triphosphates (NTPs), and treating the solution with an A-family DNA polymerase mutant or variant thereof.

In one embodiment, the invention provides a method of sequence-specific RNA synthesis comprising the steps of: providing a single-stranded DNA template in a solution of divalent cations and NTPs, providing a complementary or partially complementary annealed RNA or DNA strand as a primer, and treating the solution with an A-family DNA polymerase mutant or variant thereof.

In one embodiment, the invention provides a method of extending DNA/DNA primer-templates with one or more 5′ single-strand DNA overhang, comprising the steps of: providing a DNA/DNA primer-template in a solution of divalent cations and deoxynucleotide triphosphates (dNTPs), and treating the solution with an A-family DNA polymerase mutant or variant thereof.

In another embodiment, the invention provides a method of extending RNA/DNA primer-templates with a 5′-single-stranded DNA overhang, wherein the RNA acts as a primer for RNA synthesis, comprising the steps of: providing an RNA/DNA hybrid primer-template in a solution of divalent cations and NTPs, and treating the solution with an A-family DNA polymerase mutant or variant thereof. In some embodiments, the RNA acting as the RNA primer is a chemically modified RNA. In some embodiments, the chemical modification is one or more selected from the group consisting of a base modification, a ribose modification, a phosphate modification, a 2′-ribose modification, and a 3′-ribose modification.

In various embodiments, the NTPs or dNTPs in solution are chemically modified NTPs or dNTPs. In some embodiments, the chemical modification comprises one or more selected from the group consisting of: ribose modifications, deoxyribose modifications, base modifications, phosphate modifications, alpha-phosphate modifications, and alpha-thiophosphate modifications. In some embodiments, the NTPs or dNTPs are non-canonical NTPs.

### Applications

The RNA oligonucleotide or extended DNA or RNA nucleic acid primer-template composition of the present invention may be used in a wide variety of protocols and technologies. For example, in certain embodiments, the RNA oligonucleotide or primer-template is used in the fields of molecular biology, genomics, transcriptomics, epigenetics, nucleic acid synthesis, nucleic acid sequencing, and the like. That is, the RNA oligonucleotide or primer-template may be used in any technology that may require or benefit from the synthesis of long sequence-specific RNA oligonucleotides, modified long sequence-specific RNA oligonucleotides, or DNA or RNA primer-templates.

This method can be used in many technology platforms, including but not limited to microarray, bead, and flow cytometry. The method will be useful in numerous applications, such as genomic research, drug target validation, drug discovery, diagnostic biomarker identification and therapeutic assessment.

The method can additionally be used for rapid preparation of therapeutic RNA molecules. Such RNA molecules include, but are not limited to, antisense oligonucleotides (ASOs), short hairpin RNAs (shRNAs), small interfering RNAs (siRNAs), siRNA precursors, single guide RNAs (sgRNAs), RNA aptamers, and ribozymes.

### Kits

The present invention also relates to a kit for performing any of the methods described elsewhere herein, wherein the kit comprises one or more of: (a) an A-family polymerase (b) a reaction solution and optionally, (c) NTPs, dNTPs, modified NTPs, and/or modified dNTPs.

In one embodiment, the kit comprises an A-family polymerase. In another embodiment, the A-family polymerase is a Polθ The kit may additionally also comprise a nucleotide mixture and (a) reaction buffer(s). In certain embodiments, the kit includes a reaction buffer comprising 10 mM Mg²⁺, 40 mM Tris HCl pH 7.5, 10% glycerol, 0.01% NP-40, and 0.1 mg/mL BSA.

In particular embodiments, the kit additionally comprises one or more pre-quantified calibrator nucleic acids.

In some embodiments, one or more of the components are premixed in the same reaction container.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out exemplary embodiments of the present invention, and are not to be construed as limiting in any way the remainder of the disclosure.

### Example 1: Promoter-Independent Synthesis of Chemically Modified RNA by Human DNA Polymerase Theta Variants

The present invention is related in part to a promoter-independent DNA-dependent RNA polymerase (RNAP) with the ability to accommodate various ribonucleotide analogs and chemically modified RNA primers. A similar strategy aimed at converting A-family Thermus aquaticus (Taq) DNA polymerase (DNAP) into a DNA-dependent RNAP via a steric-gate mutation previously failed owing to the enzyme's inability to fully extend A-form RNA/DNA which is significantly wider than B-form DNA/DNA (Ong, J. L., et al., 2006, J Mol Biol, 361:537-550). For example, although conversion of the characterized steric-gate residue Glu615 to glycine—known to reduce discrimination against ribonucleoside incorporation—enabled the enzyme to efficiently incorporate ribonucleotides, the enzyme failed to synthesize RNA greater than 6-7 nt in length using a DNA/DNA primer-template substrate in the presence of Mg²⁺ (Ong, J. L., et al., 2006, J Mol Biol 361:537-550. Addition of Mn²⁺ allowed for slightly further extension of a DNA primer in the presence of ribonucleotides. These results indicated that Taq DNAP is unable to fully accommodate A-form RNA-DNA which is wider than B-form DNA-DNA.

The related A-family DNA polymerase theta (Polθ) fully extends A-form DNA/RNA primer-templates due to a significant thumb subdomain conformational change that enables accommodation of the wider DNA/RNA hybrid (Chandramouly, G., et al., 2021, Sci Adv, 7 (24): abf1771). Based on these findings, it is proposed that a steric gate Polθ mutant (E2335G; referred to as PolθRP1) would perform efficient promoter-independent RNA synthesis. The promiscuous nature of Polθ, for example use of non-canonical nucleotides, also suggested that PolθRP1 would incorporate various chemically modified ribonucleotides (Kent, T., et al., 2016, Elife, 5: e13740).

The Polθ variants described herein containing steric-gate mutations rapidly synthesize relatively long (>90 nt) sequence-specific RNA products by initiating RNA synthesis on either DNA/DNA or RNA/DNA primer-templates containing 5′ single-strand DNA (ssDNA) overhangs in vitro. These engineered enzymes synthesize long sequence-specific RNA products containing various ribonucleotide analogs commonly used for stabilizing RNA in cells for therapeutic and genome engineering applications.

Here, the ability of Polθ variants containing steric-gate mutations to rapidly synthesize relatively long (>90 nt) sequence-specific RNA products by initiating RNA synthesis on either DNA/DNA or RNA/DNA primer-templates with 5′ ssDNA overhangs is characterized in vitro. The ability of these engineered enzymes to synthesize long sequence-specific RNA products containing various ribonucleotide analogs commonly used for stabilizing RNA in cells for therapeutic and genome engineering applications is additionally demonstrated.

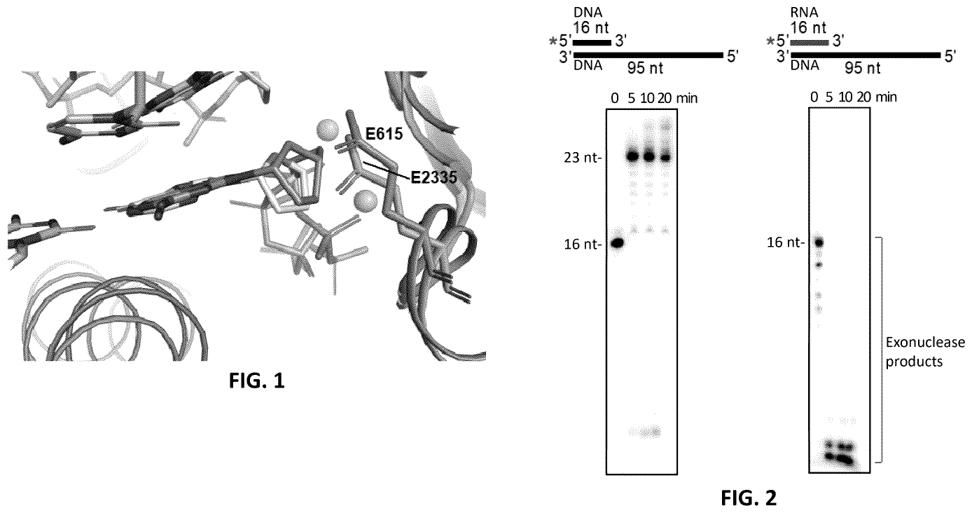

Polθ is closely related to A-family DNAPs from bacteria, such as Taq DNAP and E. coli DNAP I (Klenow fragment) with the exception of additional unstructured loops within the polymerase domain of Polθ (Black, S. J., et al., 2016, Genes, 7 (9): 67; Black, S. J., et al., 2019, Nat Commun, 10:4423; Malaby, A. W., et al., 2017, Methods Enzymol, 592:103-121; Zahn, K. E., et al., 2015, Nat Struct Mol Biol, 22:304-311). Superposition of Polθ with Taq DNAP (Li, Y., et al., 1999, Proc Natl Acad Sci USA, 96:9491-9496) is presented in FIG. 1. The superposition shows close alignment of their respective steric-gate residue which plays a major role in suppressing ribonucleotide incorporation for this A-family polymerase class. For instance, steric-gate mutants of Polθ Taq DNAP, and related E. coli Klenow fragment have been shown to efficiently incorporate ribonucleotides (Randrianjatovo-Gbalou, I., et al., 2018, Nucleic Acids Res, 46:6271-6284; Astatke, M., et al., 1998, Proc Natl Acad Sci USA, 95:3402-3407). However, steric-gate Taq DNAP and Klenow fragment mutants showed very limited RNA synthesis, resulting in pre-mature termination of RNA synthesis after incorporating ~6-7 nt on a DNA template. The observed pre-mature termination activity by steric-gate mutants of Taq DNAP and Klenow fragment in the presence of ribonucleoside triphosphates (NTPs) is due to the enzymes' inability to fully accommodate the wider A-form DNA/RNA.

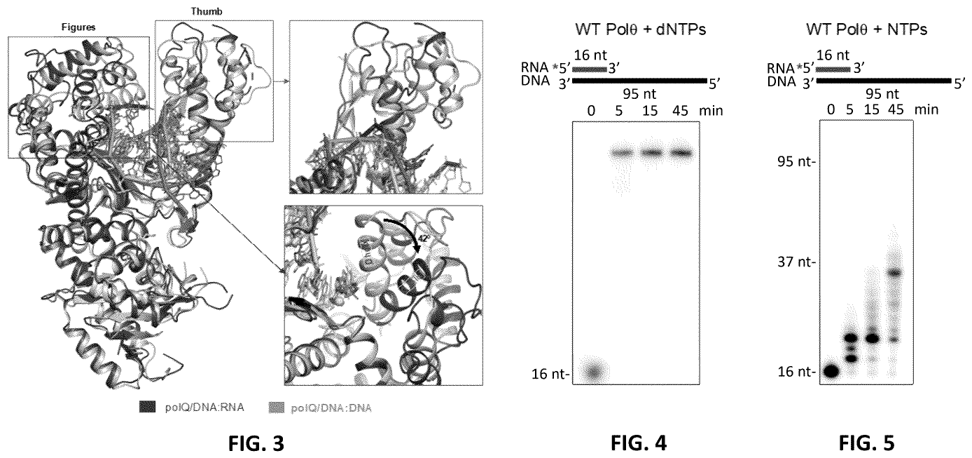

The ability of a Taq DNAP steric-gate mutant (E615G) to synthesize RNA along DNA/DNA and RNA/DNA primer-templates was tested. Consistent with prior studies, a Taq DNAP steric-gate mutant (E615G) efficiently incorporated ribonucleotides on DNA/DNA, however, the enzyme exhibited strong termination after incorporating 6-7 nt (FIG. 2, left). The Taq DNAP E615G mutant degraded the RNA primer on the RNA/DNA template under identical conditions with NTPs, likely due to its 5′-3′ exonuclease activity (FIG. 3, right). For example, although Taq DNAP possesses a 3′-5′ proof-reading like domain, similar to Klenow fragment, its activity has been inactivated by acquired mutations (Park, Y., et al., 1997, Mol Cells, 7:419-424). Here, it was found that Taq DNAP exhibits robust nuclease activity on an RNA/DNA primer, likely due to its 5′-3′ exonuclease activity. Taken together, these data further confirm the difficulties of converting Taq DNAP into a proficient promoter-independent DNA-dependent RNA polymerase that is capable of synthesizing relatively long RNA.

### Robust RNA Synthesis by Polθ Steric-Gate Variants

Prior studies elucidated the ability of Polθ to efficiently utilize DNA/RNA A-form primer-templates as substrates which revealed its ability to perform reverse transcriptase activity, similar to retroviral reverse transcriptases (Chandramouly, G., et al., 2021, Sci Adv, 7 (24): abf1771). Structural analysis of Polθ bound to a DNA/RNA primer-template compared to a DNA/DNA primer-template is reviewed in FIG. 3. The superposition of Polθ bound to DNA/RNA over the prior Polθ DNA/DNA ternary complex revealed that the thumb subdomain of Polθ undergoes an unprecedented structural rearrangement in order to accommodate the wider A-form DNA/RNA hybrid in its active center. Approximately 57% of the thumb subdomain residues were converted from alpha helices to loops. The superposition suggests that lack of this conformational change would result in a clash between the thumb subdomain and the wider RNA/DNA hybrid. The ability of Polθ to fully accommodate DNA/RNA A-form nucleic-acid in an active configuration suggests that Polθ steric-gate variants may utilize RNA/DNA primer-template substrates and synthesize long sequence-specific RNA products in a DNA template dependent manner. Furthermore, Polθ has been shown to be highly permissive in incorporating non-canonical nucleotides (Kent, T., et al., 2016, Nucleic Acids Res, 44:9381-9392). Thus, the polymerase can also accommodate various ribonucleotide analogs that are used for increasing the half-life of RNA in cells. The superposition of the two structures also revealed a significant 42-degree rotation of the fingers domain outward in the Polθ DNA/RNA complex, indicating that the enzyme was captured in an open or partially open configuration (FIG. 3).

The ability of WT Polθ (polymerase domain: residues to 1792-2590) was investigated to incorporate deoxyribonucleoside monophosphates (dNMPs) on an RNA/DNA primer-template where the primer was composed of RNA. In this scenario, the enzyme must initiate DNA synthesis on A-form RNA/DNA. As predicted from prior studies of Polθ on an A-from DNA/RNA primer-template, efficient activity of the wild-type enzyme on the RNA/DNA primer-template was observed, resulting in DNA-dependent DNA synthesis initiating from an RNA primer (FIG. 4). Hence, these data demonstrate the ability of Polθ to extend an RNA primer annealed to a DNA template and confirm the ability of the enzyme to function on A-form nucleic acid. Next, the ability of WT Polθ to incorporate ribonucleotides on the same RNA/DNA template was investigated. The WT enzyme showed inefficient DNA-dependent RNA synthesis activity which confirms its ability to strongly discriminate against ribonucleotides, like most DNAPs (FIG. 5).

To significantly reduce Polθ's discrimination against incorporating ribonucleotides, the steric-gate residue E2335 was mutated. Two different mutant versions of Polθ were generated. The first, included a single mutation in the steric gate (PolθRP1; E2335G). The second variation (PolθRP2; E2335G, I2326F) included the steric gate mutation in addition to a second mutation (I2326F) predicted to further increase the enzyme's promiscuity based on prior studies with Taq DNAP mutants. A previous study showed efficient ribonucleotide incorporation by Polθ steric-gate variants on single-strand DNA substrates. However, Polθ steric-gate mutant activity on primer-templates had not been investigated.

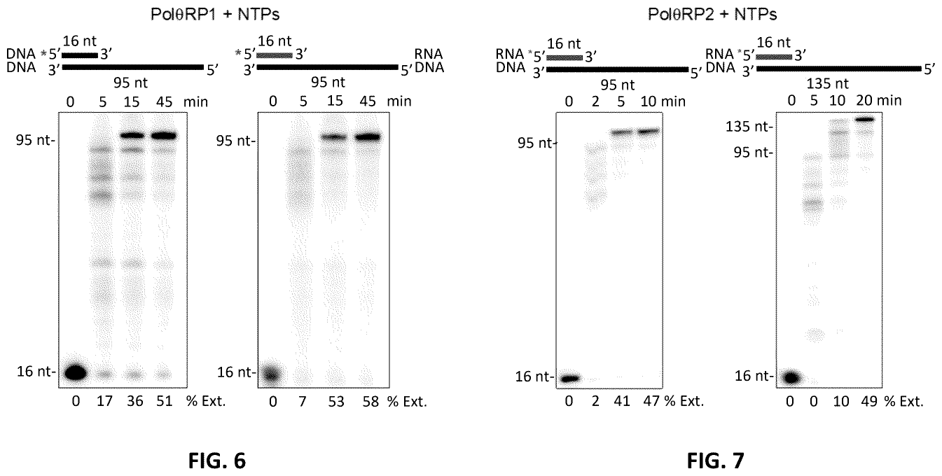

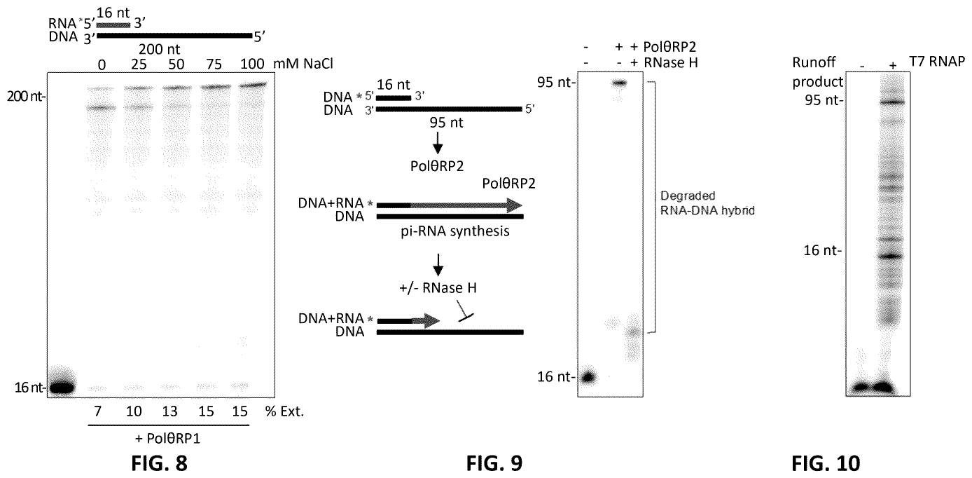

In contrast to the equivalent steric-gate mutation in Taq DNAP, PolθRP1 (E2335G) efficiently extended DNA/DNA and RNA/DNA in the presence of NTPs, resulting in highly pure RNA 95 nt in length (FIG. 6). The double mutant PolθRP2 also showed efficient RNA synthesis with high purity on RNA/DNA primer-templates, resulting sequence-specific RNA products 95 nt and 135 nt in length (FIG. 7). PolθRP1 synthesis of a 200 nt RNA is also demonstrated (FIG. 8). Here, the addition of NaCl promoted full-length sequence-specific RNA product formation. RNase H treatment following the synthesis reaction with NTPs unequivocally demonstrated that PolθRP2 synthesizes RNA as expected (FIG. 9). As a comparison, T7 RNAP synthesis of the identical 95 nt RNA sequence by initiating from its canonical promoter was highly impure due to its inherent abortive RNA synthesis activity during incomplete transition from transcription initiation to elongation phase (FIG. 10). This demonstrates a major drawback for synthesizing pure RNA oligonucleotides with T7 RNAP. These data demonstrate Polθ steric-gate mutants as effective promoter-independent DNA-dependent RNAPs and confirm that Polθ is highly active on A-form RNA/DNA.

### Comparison of Polθ and T7 RNAP on RNA/DNA Primer-Templates

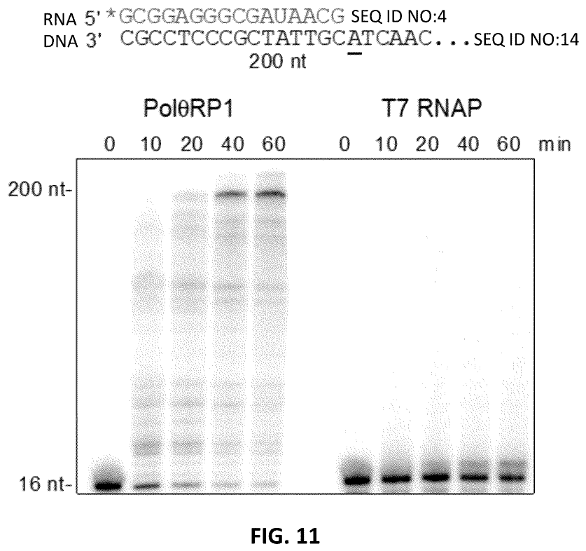

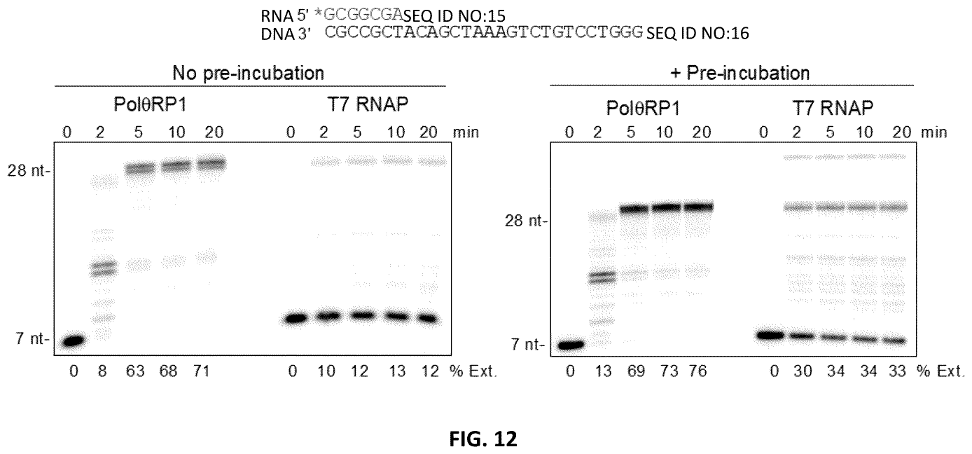

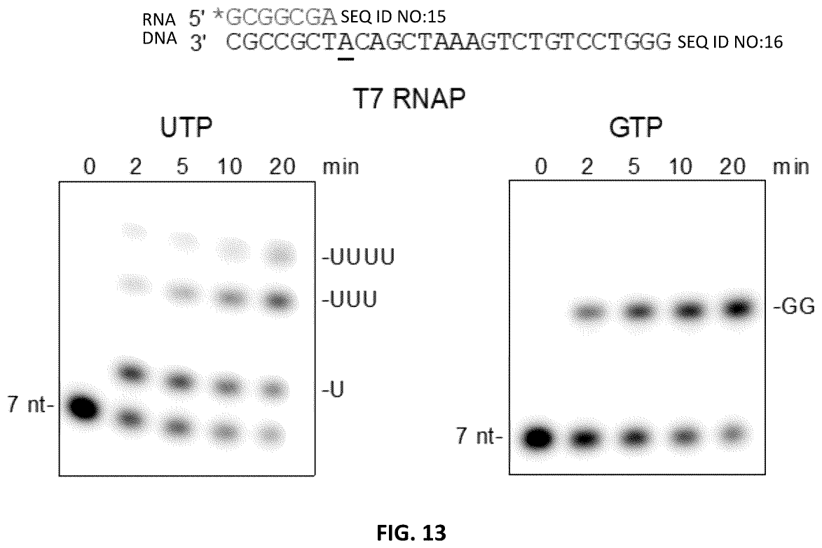

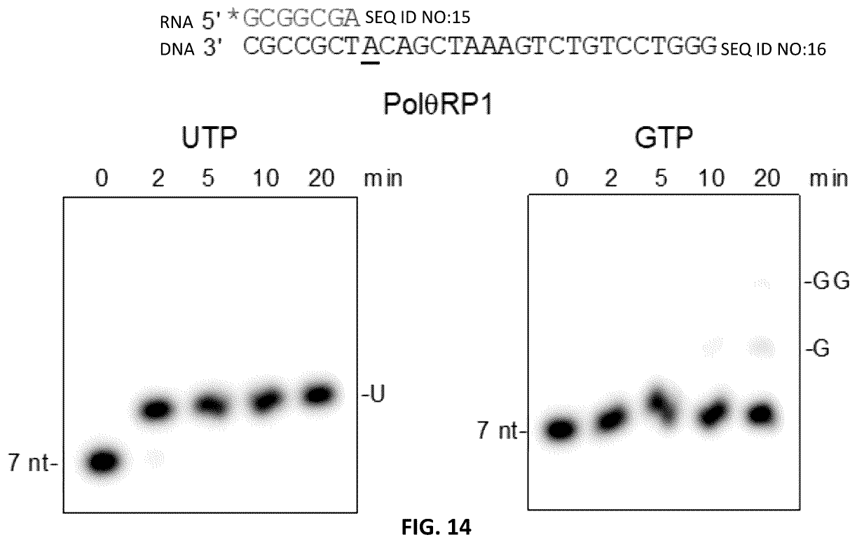

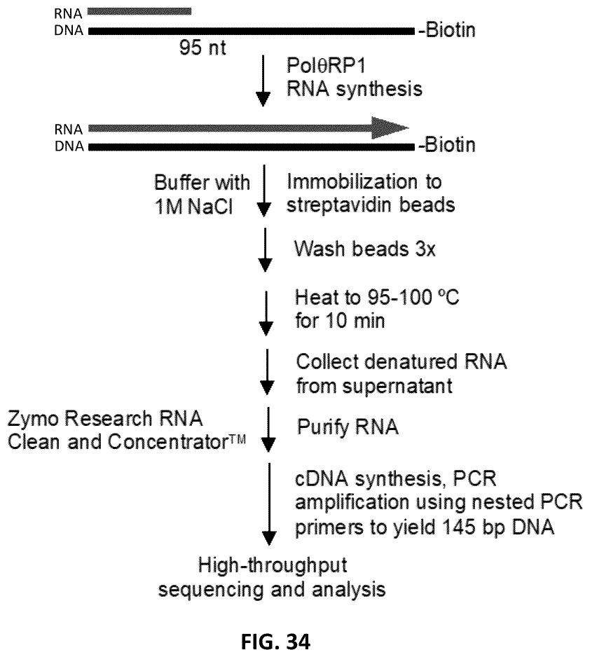

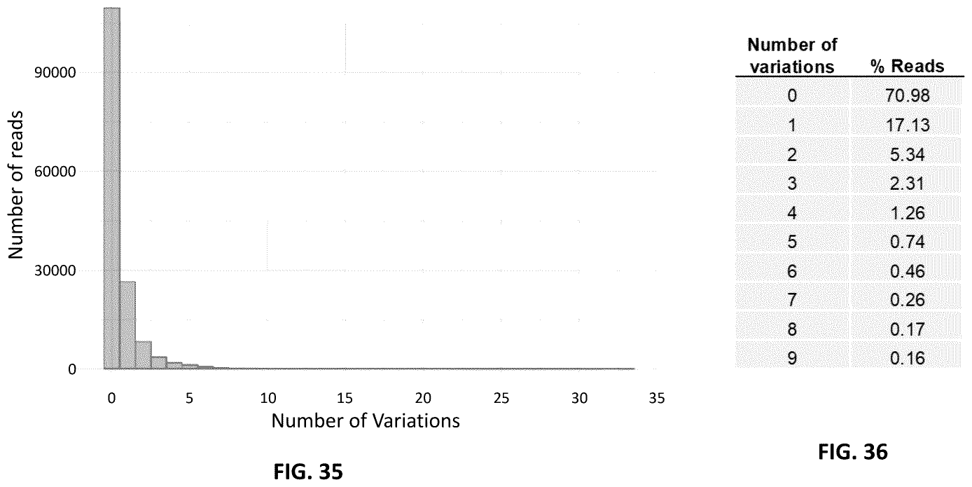

Several studies have shown the ability of T7 RNAP to form an elongation complex (EC) on short RNA/DNA primer-templates and these complexes have been used to study the biochemistry, fidelity, and structure biology of the T7 RNAP EC (Pomerantz, R. T., et al., 2006, Mol Cell, 24:245-255; Tahirov, T. H., et al., 2002, Nature, 420:43-50). Considering that T7 RNAP is used to synthesize mRNA vaccines and RNA for basic research, this prototypical promoter-dependent RNAP was further compared to PolθRP1 on an RNA/DNA primer-template using identical conditions. T7 RNAP was inactive compared to PolθRP1 on an RNA/DNA primer-template comprised of a 16 nt RNA and 200 nt DNA template (FIG. 11). Reducing the RNA primer length to 7 nt on a shorter DNA template allowed minor (10-13%) RNA extension activity by T7 RNAP compared to PolθRP1 (FIG. 12, left). Next, both enzymes were pre-incubated on the RNA/DNA primer-template for 10 min prior to the addition of NTPs since T7 RNAP is known to form an EC under these conditions. T7 RNAP was still deficient in extending the RNA/DNA template even after 20 min, whereas PolθRP1 showed nearly full extension after 5 min (FIG. 12, right). Next, the relative efficiencies of ribonucleotide misincorporation by PolθRP1 and T7 RNAP were compared. Prior studies have shown the ability to measure the fidelity of the T7 RNAP EC on short RNA/DNA primer-templates. Thus, similar conditions were used to compare correct (UMP) versus incorrect (GMP) ribonucleotide incorporation by T7 RNAP EC following the necessary 10 min pre-incubation period. Consistent with prior studies, T7 RNAP misincorporated two GMPs via a template strand misalignment mechanism (FIG. 13, right). Also consistent with prior studies, after T7 RNAP correctly incorporated UMP, it proceeded to misincorporate UMP (FIG. 13, left). Using identical conditions, it was unexpectedly found that PolθRP1 showed little to no capacity to misincorporate GMP (FIG. 14, right). PolθRP1 showed no ability to misincorporate UMP after the initial correct incorporation step (FIG. 14, left). These findings suggest PolθRP1 exhibits higher fidelity DNA-template dependent RNA synthesis compared to T7 RNAP and are consistent with prior studies demonstrating that PolθRP1 exhibits higher fidelity on A-form versus B-form primer-templates. High-throughput sequencing and analysis of PCR amplified cDNA synthesized from PolθRP1 sequence-specific RNA products showed that 71% of sequencing reads aligned perfectly to the reference sequence, whereas 17% of the reads had only 1 base variation (FIGS. 34-36). Error-prone reverse transcriptase was used to synthesize the cDNA for this RNA sequencing method. Thus, this method is likely biased towards a higher error-rate.

### Polθ Variant Synthesis of Chemically Modified RNA

Chemically modified ribonucleotide analogs enable stabilization of RNA in cells and reduce immunogenicity. For example, pseudouridine significantly increases the half-life of synthetic mRNA by reducing its immunogenicity and enzymatic cleavage by RNase L, and later developments led to the use of N1-methylpseudouridine in mRNA vaccines (Zhao, B. S., et al., 2015, Cell Res, 25:153-154; Kariko, K., et al., 2012, Mol Ther, 20:948-953; Kariko, K., et al., 2008, Mol Ther, 16:1833-1840; Anderson, B. R., et al., 2011, Nucleic Acids Res, 39:9329-9338; Nance, K. D., et al., 2021, ACS Cent Sci, 7:748-756). Hence, this single chemical modification in synthetic RNA may be among the most important developments in modern RNA biotechnology. Additional naturally occurring ribonucleotides in cellular RNA include 5-methyl-cytidine and N6-methyl-adenosine, among many others (McCown, P. J., et al., Wiley Interdiscip Rev RNA, 11: e1595; Zhang, C., et al., 2019, Front Immunol, 10:922; Boo, S. H., et al., 2020, Exp Mol Med, 52:400-408).

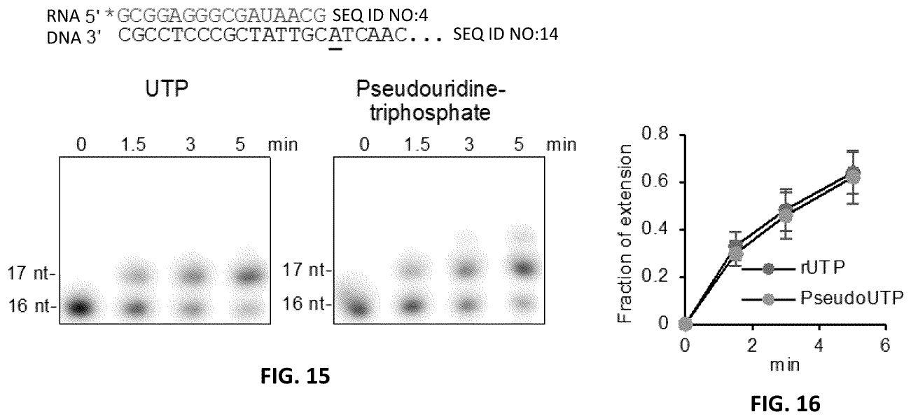

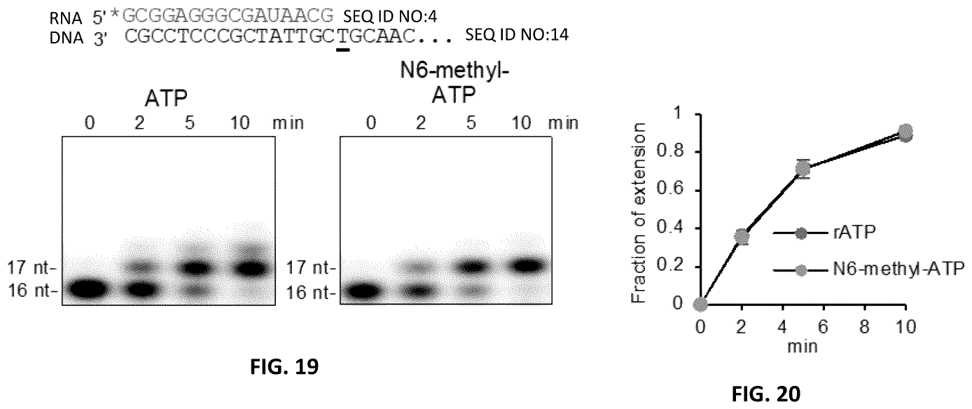

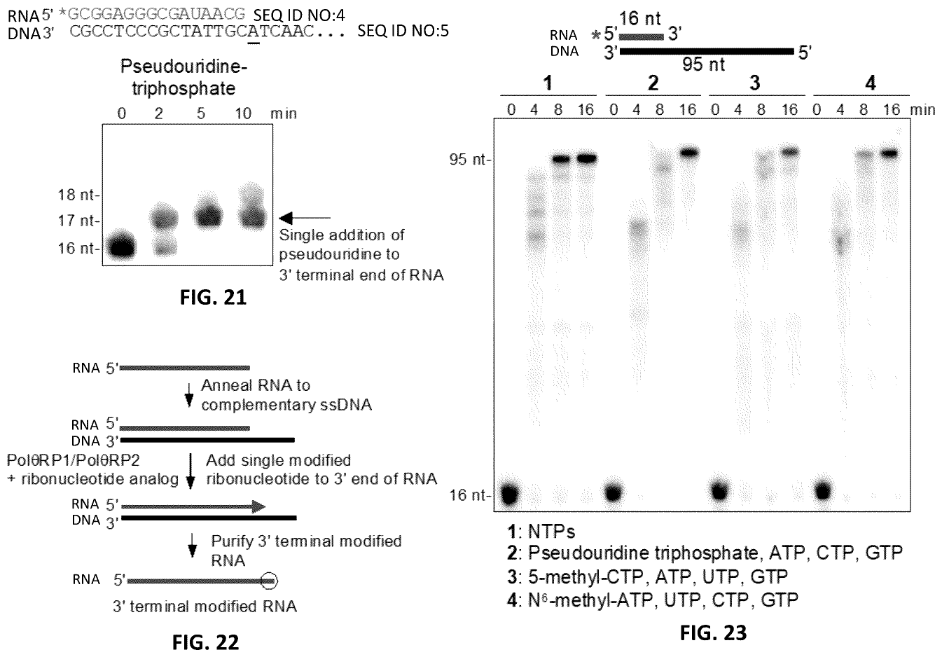

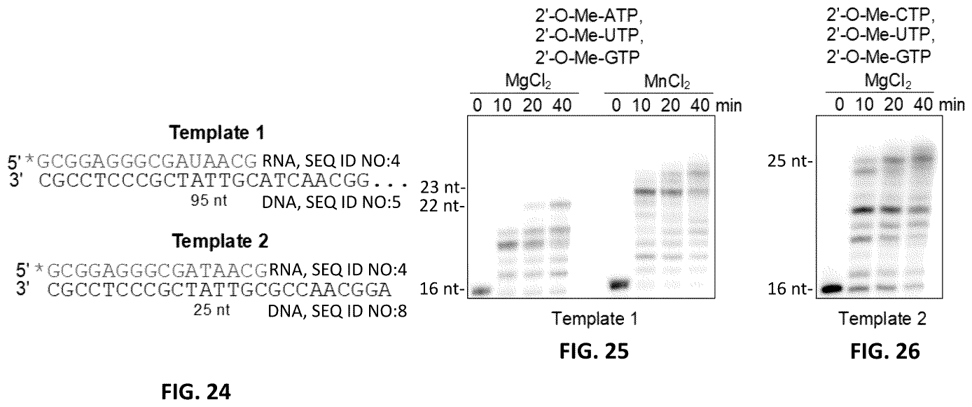

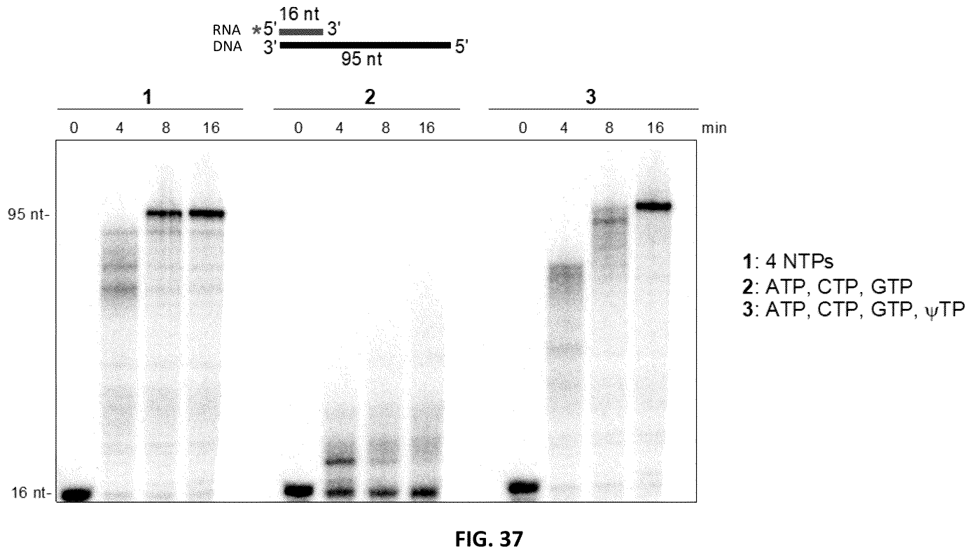

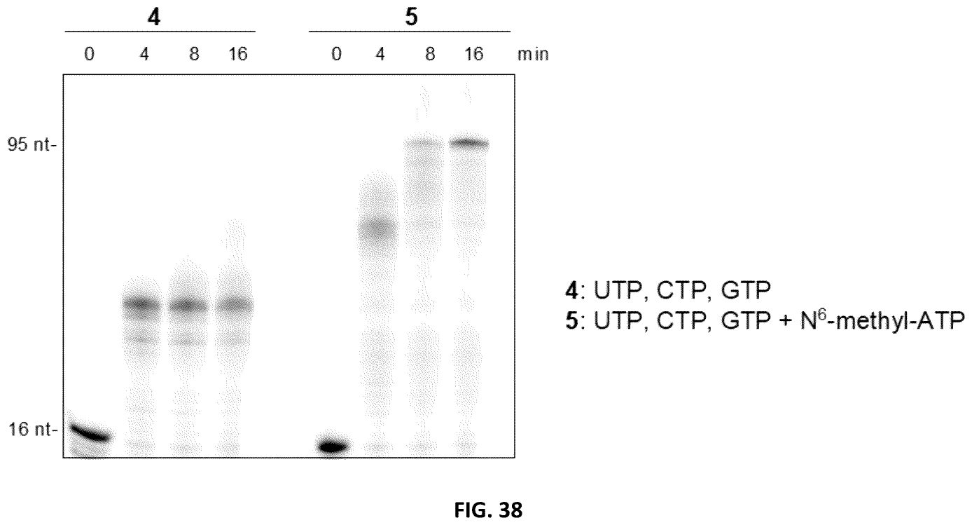

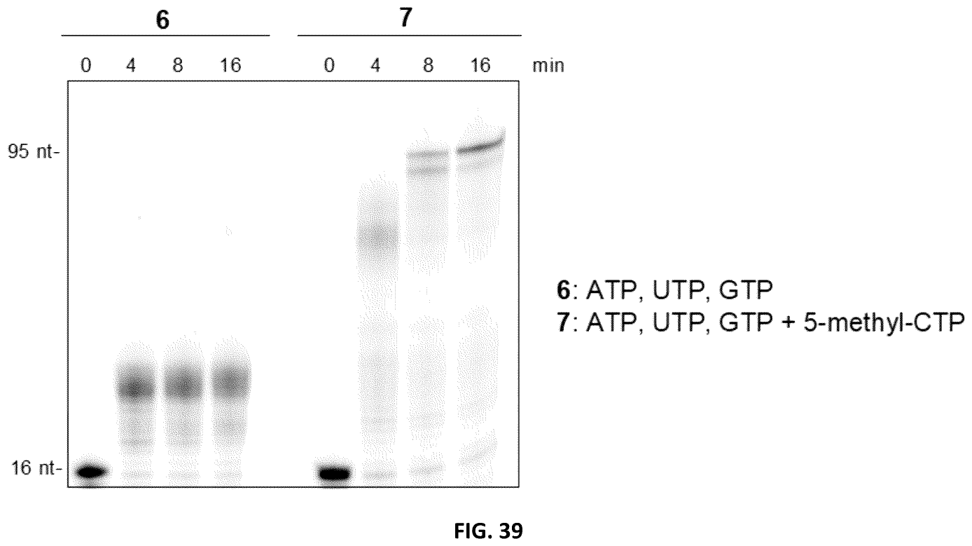

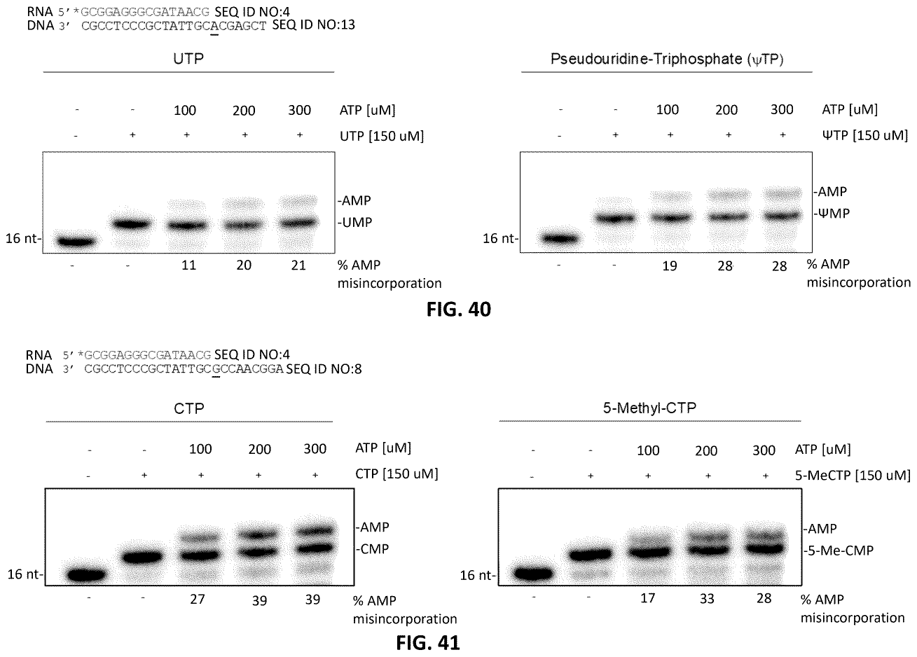

PolθRP2 efficiently incorporates pseudouridine, 5-methyl-cytidine, and N6-methyl-adenosine monophosphates with similar efficiency as canonical ribonucleotides (FIGS. 15-20). Importantly, time-dependent extension of RNA/DNA with nucleotide analogs enabled incorporation of single nucleotide analogs, resulting in specific 3′-terminal RNA chemical modifications. For example, the right panels in FIGS. 15, 17, and 19 shows the ability to incorporate single ribonucleotide analogs (pseudouridine, 5-methyl-cytidine, N6-Methyl-adenosine) in a time-controlled manner. Further experimentation with pseudouridine triphosphate revealed 5 min as optimal for incorporating a single pseudouridine monophosphate at the 3′ terminal end of RNA (FIG. 21). The ability of PolθRP2 to incorporate single modified ribonucleotides in a time dependent manner enables rapid modification of the 3′ terminal end of RNA molecules via enzymatic activity. For example, following annealing of the 3′ end of RNA to a complementary ssDNA oligonucleotide allows for single ribonucleotide incorporation by Polθ steric gate mutants (FIG. 22). The modified RNA species may then be purified and used for downstream biotechnology applications. It is also possible to add multiple consecutive chemically modified ribonucleotide analogs in a template and time dependent manner at the 3′ terminal end of RNA which will be useful for reducing nuclease activity or immunogenicity of RNA used for therapeutic and genome engineering applications. Because PolθRP2 exhibits efficient incorporation of base modified ribonucleotide analogs, it was investigated whether the mutant enzyme can synthesize relatively long RNA with substitution of a canonical ribonucleotide with a base modified analog. The results show that PolθRP2 exhibits efficient synthesis of highly pure RNA in the presence of pseudouridine triphosphate, 5-methyl-CTP and N6-methyl-ATP substituted for UTP, CTP and ATP, respectively (FIG. 23). Controls show that the Polθ steric gate variant fails to synthesize these RNA products when only three NTPs are added to the reaction (FIGS. 37-39), consistent with a poor capacity to incorporate incorrect ribonucleotides as shown in FIG. 14. Additional controls show that PolθRP1 does not show a preference for misincorporation during primer extension with NTP analogs (FIGS. 40 and 41). Taken together, the results presented in FIGS. 15-23 demonstrate the ability of the Polθ steric gate variant to efficiently incorporate ribonucleotide analogs with modified base moieties and synthesize RNA with base modifications.

Various 2′ ribonucleoside modifications are widely used at the terminal ends of RNA based antisense therapeutics for their ability to enhance complementary strand binding via higher Tm, reduce immunogenicity, and suppress nuclease digestion (Khvorova, A., et al., 2017, Nat Biotechnol, 35:238-248). Polθ steric-gate mutants appear to utilize 2′-O-Methyl NTPs with relatively low efficiency. For example, PolθRP1 exhibited slow RNA extension in the presence of three 2′-O-methyl NTPs and showed strong pausing or premature termination events (FIG. 25, left). The addition of MnC₂ instead of MgCl₂ improved the rate of extension with 2′-O-methyl NTPs (FIG. 25, right). Polymerases have been shown to exhibit more promiscuous activity in the presence of MnCl₂. PolθRP1 activity with 2′-O-methyl NTPs was examined in a different sequence context in FIG. 26. Significant pausing and/or premature termination events are also observed on this template, demonstrating that Polθ steric-gate mutants exhibit limited capacity to synthesize RNA with consecutive 2′-O-methyl ribose modifications.

Some biotechnology applications may require RNA to be fully composed of modified ribonucleotides for superior stability and/or cellular uptake. For example, phosphorothioate modification of RNA is known to enhance the stability of synthetic RNA against nucleases and improve RNA uptake (Khvorova, A., et al., 2017, Nat Biotechnol, 35:238-248). The phosphorothioate linkage represents a sulfur replacement of a nonbridging phosphodiester oxygen and is one of the most widely used nucleic acid modifications (Shen, X., et al., 2018, Nucleic Acids Res, 46:1584-1600). Along with 2′-O-methyl and 2′-O-methoxyethyl modifications, phosphorothioate modifications are among the most frequently used chemical modifications for RNA based therapeutics in clinical trials. Furthermore, it was recently shown that phosphorothioate mRNA modification accelerates the translation initiation rate, resulting in higher efficiency of protein synthesis. Thus, phosphorothioate modified mRNA is likely to increase the efficacy of mRNA vaccines (Kawaguchi, D., et al., 2020, Angew Chem Int Ed Engl, 59:17403-17407).

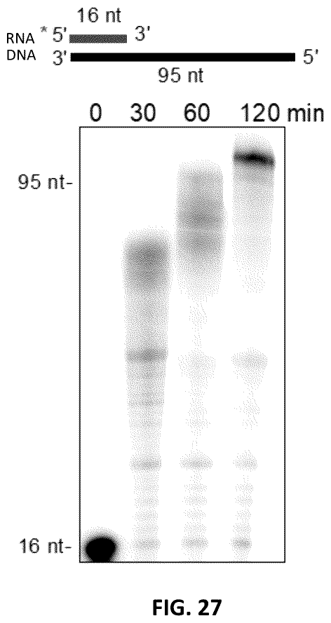

Notably, PolθRP1 mediated full-length template-dependent RNA synthesis in the presence of all four α-phosphorothioate-NTPs, although the rate of RNA synthesis was significantly slower compared to canonical NTPs (FIG. 27). For example, although the enzyme was able to fully extend the RNA primer in the presence of all four α-phosphothio-NTPs, the full-length product was not observed for at least 60 min at 37° C. The reduced RNA synthesis rate may be due to slower phosphodiester bond formation in the presence of α-phosphothio-NTPs.

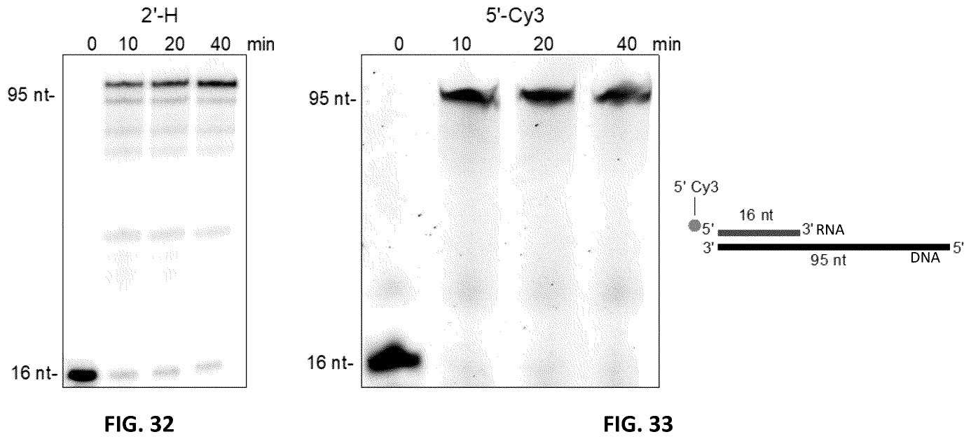

As a strategy to enable rapid synthesis of relatively long sequence-specific RNA with 5′-terminal 2′ ribose chemical modifications, extension of 5′ modified RNA primers by PolθRP1 was examined in the presence of canonical NTPs. The results show that PolθRP1 efficiently synthesizes sequence-specific RNA products by extending RNA primers containing multiple consecutive 2′ ribonucleotide modifications (2′-O-Me, 2′-MOE (2′-O—CH₂—CH₂—O—CH₃), 2′-F) at the 5′ terminus (FIGS. 29-31). The mutant enzyme is able to synthesize sequence-specific RNA with a DNA-RNA chimeric primer containing 6 consecutive deoxyribonucleotides at the 5′ terminus (FIG. 32). PolθRP1 also extends an RNA primer containing a 5′ terminal Cy3 fluorophore. In this case the RNA was visualized via Cy3 fluorescence imaging (FIG. 33). These data demonstrate the utility of synthesizing relatively long RNA with various 5′ terminal modifications which may be useful for biotechnology and biomedical applications, and basic RNA research.

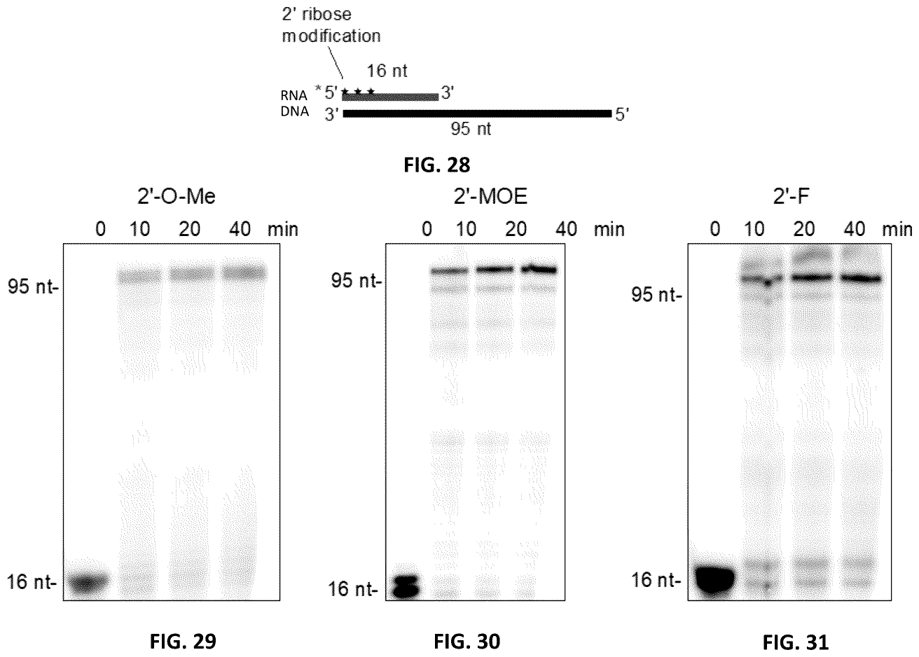

This report demonstrates rapid promoter-independent enzymatic synthesis of long sequence-specific RNA oligonucleotides with canonical NTPs and various ribonucleotide analogs using engineered Polθ steric-gate mutants by extending pre-annealed DNA/DNA and RNA/DNA primer-templates. The results presented herein demonstrate the ability to enzymatically synthesize synthetic RNA containing site-specific chemical modifications by using two strategies. The first enables 5′-terminal modifications by utilizing a synthetic RNA primer with 5′ terminal modified ribonucleotides (FIGS. 28-33). The second strategy enables the incorporation of 3′-terminal ribonucleotide modifications by annealing synthetic RNA to a sequence specific DNA template and incorporating a specific number of ribonucleotides in a time dependent manner (FIG. 22). These enzymatic mechanisms and strategies pave the way for next-generation RNA oligonucleotide synthesis methods. For example, automation and high-throughput implementation of the methods described herein, and related enzymatic methods, have the potential to lower the production costs of synthetic RNA with 5′ and 3′ chemical modifications, increase yields, and reduce turnaround time. Specifically, the use of liquid handler-based robotics and nucleic acid surface attachment methods can conceivably enable repeated use of immobilized DNA templates which can potentially increase the yield of enzymatic synthesis of RNA oligonucleotides. Hence, an automated solid-phase enzymatic RNA synthesis platform based on this technology can conceivably be scaled up for kilogram scale production of synthetic RNA for antisense and genome engineering applications. Additionally, high-throughput development of this RNA synthesis technology can possibly enable rapid synthesis of RNA oligonucleotide libraries for discovery-based research. Finally, engineering additional classes of promoter-independent DNA-dependent RNAPs with distinct ribonucleotide substrate specificities and enzyme characteristics may broaden the capabilities of promoter-independent enzymatic RNA synthesis for the production of chemically modified synthetic RNA oligonucleotides for anti-sense therapeutics and genome engineering applications. For instance, novel bioengineered promoter-independent DNA-dependent RNAPs that exhibit higher efficiency of incorporating ribonucleotides with 2′-OH modifications may be beneficial for the production of highly stable synthetic RNA for therapeutics and genome engineering.

### Materials and Methods

Proteins: All site-directed mutations were generated using Agilent QuikChange II following manufacturers protocol.

WT Polθ PolθRP1 and PolθRP2 were expressed and purified as described (Hogg, M., et al., 2011, J Mol Biol, 405:642-652), with the following changes. The eluted fractions from 5 ml His-Trap column (Cytiva) were diluted to 180 mM NaCl, loaded onto a 5 ml Heparin Hi-Trap column (Cytiva), and eluted with a gradient to buffer C (50 mM Hepes 8.0; 10% Glycerol; 0.005% NP-40) containing 1 M NaCl. Fractions with target protein were pooled, mixed with 10 units of suitable SUMO protease (LifeSensors), and dialyzed overnight against buffer C containing 300 mM NaCl and 20 mM β-mercaptoethanol. The digested fractions were then further purified over a HisTrap column by separating the cleaved His-tag and undigested protein fraction.

T7 RNAP was purified as described (McDevitt, S., et al., 2018, Nat Commun, 9:1091).

Purification of Taq DNAP E615G. pET28a vector (Addgene) expressing N-terminally 6HIS-tagged version of the Taq DNAP E615G was transformed into BL21 (DE3) cells (Invitrogen). The E615G mutation was generated via site-directed mutagenesis using QuikChange II XL Site-Directed Mutagenesis Kit (Agilent). Freshly grown colonies were inoculated into a starter culture of 40 mL LB supplemented with 50 μg/mL kanamycin and were shaken overnight at 37° C. Next, the overnight cells were added to 4 L of LB with 50 μg/mL kanamycin and grown at 37° C. until OD₆₀₀ ~0.5. Then the shaker temperature was turned to 18° C., and the cells were growing for the next 1 hour followed by addition of IPTG to a final concentration of 0.2 mM. The cells were further shaken overnight, next pelleted in a centrifuge at 4° C. (30 min at 3,000 g) and frozen at −80° C. Frozen pellets (20 g) were thawed on ice and resuspended in 200 mL of lysis buffer containing 50 mM HEPES pH 8.0, 0.5 M NaCl, 10% glycerol, 10 mM imidazole pH 8.0, 5 mM BME, 1.5% Igepal CA630 supplemented with 2 mM PMSF and 4 tablets of SIGMAFAST EDTA-free protease inhibitor cocktail (Sigma). The cells were sonicated on ice, heated in a water bath for 40 min at 65° C. and centrifuged for 60 min at 25,000 g. The cleared lysate was loaded onto a 5 mL HisTrap FF crude column (Cytiva) and washed with buffer A (50 mM HEPES pH 8.0, 0.5 M NaCl, 10% glycerol, 35 mM imidazole pH 8.0, 5 mM BME, 0.005% Igepal). The bound protein was eluted with buffer B containing 200 mM imidazole. The fractions containing Taq DNAP were pooled and dialyzed against 1 L of buffer C (50 mM HEPES pH 8.0, 0.1 M NaCl, 10% glycerol, 5 mM BME, 0.005% Igepal) overnight at 4° C. The protein was then loaded onto a 5 mL Hi Trap Heparin HP column (Cytiva) and eluted with a NaCl gradient (from 0.1 M to 1 M) in the buffer C. Fractions containing Taq DNAP were pooled, concentrated on a spin concentrator Amicon Ultra with 30,000 MWCO (Sigma), centrifuged 10 min at 20,000 g and loaded onto a size exclusion column Superdex200 Increase 10/300 (GE Healthcare). The desired protein fractions were combined, aliquoted and frozen at −80° C.

Nucleic acids: Primer strands were 5′-phosphorylated with T4 polynucleotide kinase (New England Biolabs) and (γ-³²P) ATP (Perkin Elmer) in 1X T4 polynucleotide kinase buffer (New England Biolabs) at 37° C. for 60 min. DNA/DNA and RNA/DNA Primer templates were annealed by mixing a ratio of 1:1.5 of primer to template then heating to 95-100° C. followed by slow cooling to room temp. The primer in FIG. 7F was 5′ conjugated with a Cy3 fluorophore. All oligonucleotides were purchased from Integrated DNA Technologies (IDT) and sequences (5′-3′) are listed herein.

RP635:
(SEQ ID NO: 3)
GCGGAGGGCGATAACG
RP635R:
(SEQ ID NO: 4)
rGrCrGrGrArGrGrGrCrGrArUrArArCrG
RP273:
(SEQ ID NO: 5)
AGACTCCGTATCGTAAAGTGACCGACGGTGTTGTAACTG
ACGAAATTCACTACCTGTCTGCTATCGAAGAAGGCAACTACGTTATCGC
CCTCCGC
RP273B:
(SEQ ID NO: 6)
/Biotin/AGACTCCGTATCGTAAAGTGACCGACGGTGTTGT
AACTGACGAAATTCACTACCTGTCTGCTATCGAAGAAGGCAACTACGTT
ATCGCCCTCCGC
RP643:
(SEQ ID NO: 7)
TCCTGGCCAATGAGATGGCAGCTGCCAATGGCTGGGCAC
ACAGACTCCGTATCGTAAAGTGACCGACGGTGTTGTAACTGACGAAATT
CACTACCTGTCTGCTATCGAAGAAGGCAACTACGTTATCGCCCTCCGC
RP644:
(SEQ ID NO: 8)
AGGCAACCGCGTTATCGCCCTCCGC
RP645:
(SEQ ID NO: 9)
AGGCAACGTCGTTATCGCCCTCCGC
RP665:
(SEQ ID NO: 10)
AGGCAACGCCGTTATCGCCCTCCGC
RP668:
(SEQ ID NO: 11)
GATCGATTAATACGACTCACTATAGGGCGGAGGGCGATA
ACGTAGTTGCCTTCTTCGATAGCAGACAGGTAGTGAATTTCGTCAGTTA
CAACACCGTCGGTCACTTTACGATACGGAGTCT
RP668C:
(SEQ ID NO: 12)
AGACTCCGTATCGTAAAGTGACCGACGGTGTTGTAACT
GACGAAATTCACTACCTGTCTGCTATCGAAGAAGGCAACTACGTTATCG
CCCTCCGCCCTATAGTGAGTCGTATTAATCGATC
RP670:
(SEQ ID NO: 13)
TCGAGCACGTTATCGCCCTCCGC
TKD200:
(SEQ ID NO: 14)
CCACTTTTCAAGTTGATAACGGACTAGCCTTATTTAACT
TGCTATGCTGTTTTGAATGGTTCCCAAAACAGCATAGCTCTAAAACACA
GTTCCTGACTACGAAAGAGACTCCGTATCGTAAAGTGACCGACGGTGTT
GTAACTGACGAAATTCACTACCTGTCTGCTATCGAAGAAGGCAACTACG
TTATCGCCCTCCGC
R7:
(SEQ ID NO: 15)
rGrCrGrGrCrGrA
TS01:
(SEQ ID NO: 16)
GGGTCCTGTCTGAAATCGACATCGCCGC

### Primer Extension Assays;

FIG. 2: Primer extension assays with 100 nM of Taq DNAP E615G were incubated with 15 nM of radiolabeled DNA/DNA (RP635R/RP665) or RNA/DNA (RP635R/RP665) in buffer A (20 mM Tris-HCl pH 7.5, 0.01% NP-40, 0.1 mg/ml BSA, 10% glycerol, 10 mM MgCl₂) and 150 μM NTPs at 37° C. for 30 min.

FIGS. 4-7: Primer extension assays were performed with 50 nM WT Polθ (2B, 2C), 100 nM PolθRP1 (2D), and 100 nM PolθRP2 (2E) and were incubated with 15 nM 5′-radiolabeled RNA/DNA (RP635R/RP273) or DNA/DNA (RP635/RP273) for the indicated times in buffer A with 150 μM dNTPs (2B) or NTPs (2C, 2D, 2E) at 37° C.

FIG. 8: Primer extension assays were performed with 200 nM PolθRP1 and were incubated with 15 nM 5′-radiolabeled RNA/DNA (RP635R/TKD200) for 60 min in buffer A with the indicated concentrations of NaCl and 300 μM dNTPs at 37° C.

FIG. 9: Primer extension assays were performed with 100 nM PolθRP2 and were incubated with 15 nM of 5′-radiolabeled DNA/DNA (RP635/RP273) for the indicated times in buffer A with 150 μM NTPs at 37° C. 2 units of RNase (New England Biolabs) was added as indicated for an additional 30 min at 37° C.

FIG. 11: Primer Extensions assays with 100 nM of PolθRP-1 and T7 RNAP were incubated with 50 nM of radiolabeled RNA/DNA (RP635R/TKD200) primer/template for the indicated time in minutes within a buffer containing (0.01% NP-40, 0.1 mg/ml BSA, 10 mM MgCl₂, 10% glycerol) with 300 μM NTPs at 37° C. for the indicated time periods. PolθRP1 assays also contained 20 mM Tris at pH 7.5 and 0.5 mM DTT, while T7 RNAP assays also contained 40 mM Tris at pH 7.9 and 1 mM DTT.

FIG. 12: Primer Extensions assays with 100 nM of PolθRP1 and T7 RNAP were incubated with 50 nM of radiolabeled RNA/DNA (R7/TS01) primer/template for the indicated time in minutes within a buffer containing (0.01% NP-40, 0.1 mg/ml BSA, 10 mM MgCl₂, 10% glycerol) at 37° C. for the indicated time periods with or without a 10 minute pre-incubation at 25° C. before NTPs were added for a final concentration of 300 μM. PolθRP1 assays also contained 20 mM Tris at pH 7.5 and 0.5 mM DTT, while T7 assays also contained 40 mM Tris at pH 7.9 and 1 mM DTT.

FIGS. 13 and 14: Primer Extensions assays with 200 nM of PolθRP-1 and T7 RNAP were incubated with 100 nM of radiolabeled RNA/DNA (R7/TS01) primer/template for the indicated time in minutes within a buffer containing (0.01% NP-40, 0.1 mg/ml BSA, 10 mM MgCl₂, 10% glycerol) with 300 M the indicated nucleotides at 37° C. for the indicated time periods. PolθRP1 assays also contained 20 mM. Tris at pH 7.5 and 0.5 mM DTT, while T7 RNAP assays also contained 40 mM Tris at pH 7.9 and 1 mM DTT.

FIGS. 15 and 16: Primer extension assays were performed with 40 nM PolθRP2 and were incubated with 60 nM 5′-radiolabeled RNA/DNA (RP635R/RP273) for the indicated times in buffer A supplemented with 10 mM MgCl₂ along with 150 μM of UTP or pseudouridine-triphosphate at 25° C.

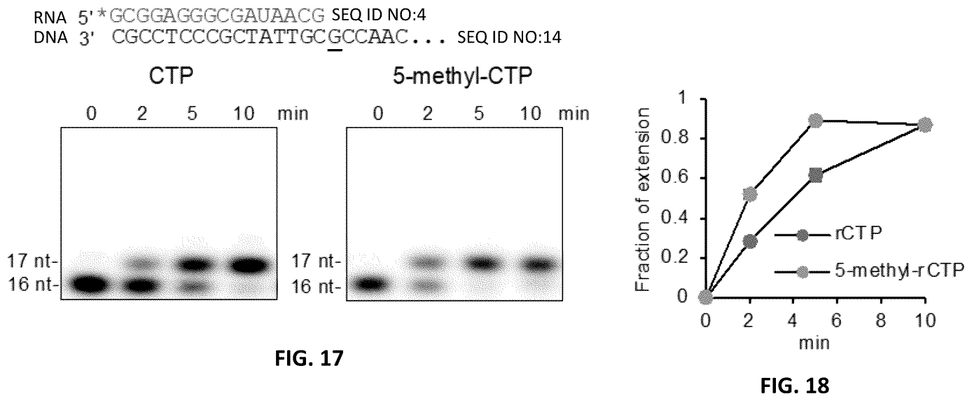

FIGS. 17-20: Primer extension assays were performed with 20 nM of PolθRP2 and were incubated with 60 nM of 5′-radiolabeled RNA/DNA (RP635R/RP644 (C,D); RP635R/RP645 (E,F)) for the indicated times in buffer A with 150 μM CTP or 5-methyl-deoxycytidine triphosphate (C,D) and 150 μM ATP or N6-methyl-adenosine triphosphate (E,F) at 25° C. Scatter plots represent avg % extension at the indicated times. Data represent mean (n=3)+S.D. Percent extension was calculated by dividing the intensity of the sum of the extended products by the sum of the intensity of the extended and unextended products then multiplying by 100. Relative gel band intensities were determined using ImageJ.

FIG. 21: Primer extension assays were performed with 40 nM PolθRP2 and were incubated with 60 nM 5′-radiolabeled RNA/DNA (RP635R/RP273) for the indicated times in buffer A with 150 μM pseudouridine-triphosphate at 25° C.

FIG. 23: Primer extension assays were performed with 100 nM PolθRP2 and were incubated with 15 nM 5′-radiolabeled RNA/DNA (RP635R/RP273) for the indicated times in buffer A with 150 μM of the indicated nucleotides at 37° C.

FIG. 25: Primer extension assays were performed with 100 nM PolθRP1 and were incubated with 20 nM of 5′-radiolabeled RNA/DNA (RP635R/RP273) for the indicated times in buffer A supplemented with 5 mM MgCl₂ or 5 mM MnCl₂ with 150 μM of the indicated 2′-O-methyl-nucleoside triphosphates at 37° C.

FIG. 26: Primer extension assays were performed with 100 nM PolθRP1 and were incubated with 20 nM 5′-radiolabeled RNA/DNA (RP635R/RP644) for the indicated times in buffer A with 150 μM of the indicated 2′-O-methyl-nucleoside triphosphates at 37° C.

FIG. 27: Primer extension assays were performed with 100 nM of PolθRP1 and were incubated with 20 nM of 5′-radiolabeled RNA/DNA (RP635R/RP273) for the indicated times in buffer A (supplemented with 5 mM MgCl₂, 1 mM MnCl₂ and 1 mM DTT) along with 150 μM of α-phosphothioate-NTPs at 37° C.

FIG. 29-32: Primer extension assays were performed with 400 nM of PolθRP1 and were incubated with 20 nM of 5′-radiolabeled RNA/DNA (RP635R/RP273 with the indicated 5′-terminal chemical modifications to the RNA primer) in buffer A for the indicated times with 250 μM of NTPs at 37° C.

FIG. 32: Primer extension assays were performed with 400 nM of PolθRP1 and were incubated with 20 nM of 5′-Cy3 conjugated RNA/DNA (RP635R-Cy3/RP273) for the indicated times in buffer A with 250 μM NTPs at 37° C.

All primer extension assays were terminated with 25 mM EDTA and 45% formamide. Radio-labeled DNA and RNA products and Cy3 conjugated RNA products were resolved in urea denaturing 15% polyacrylamide gels and visualized by phosphorimager.

### In Vitro Transcription:

FIG. 9: In vitro transcription assays were performed with 100 nM T7 RNAP and were incubated with 20 nM double-strand DNA (RP668/RP668C) for the indicated times in buffer B (40 mM Tris-HCl pH 7.9, 0.01% NP-40, 0.1 mg/ml BSA, 6 mM MgCl₂, 1 mM DTT, and 10% glycerol) with 300 μM NTPs, along with 32P-a-ATP (Perkin Elmer) at 37° C. Assays were terminated with 25 mM EDTA and 45% formamide then radio-labeled RNA was resolved in urea denaturing 15% polyacrylamide gels and visualized by phosphorimager.

RNA sequencing and analysis: RNA synthesis was performed by pre-incubating 75 nM PolθRP1 with 20 nM 635R/273B RNA/DNA primer-template in buffer 25 mM TrisHCl pH 7.8, 0.01% NP-40, 10% glycerol, 5 mM MgCl₂, 0.1 mg/ml BSA for 5 min at 37° C. RNA synthesis was initiated by adding 200 μM NTPs for an additional 30 min. The reaction was then mixed with Dynabeads™ M-280 streptavidin pre-washed with buffer (described elsewhere herein) in the presence of 1M NaCl. The RNA/DNA product was incubated with the beads for ~10 min, then the beads were washed 3 times using a magnetic holder with buffer plus 1M NaCl using 500 μl volumes to remove unbound protein and nucleic acid. Following washing, beads were suspended in 50 mM TrisHCl pH 8.8, boiled for 10 min, then placed on ice for 5 min. The supernatant was collected and nucleic acids purified using Zymo Research RNA Clean and Concentrator™ kit at room temp. Purified RNA was used for cDNA synthesis using Multiscribe™ Reverse Transcriptase (Applied Biosystems) using manufacturers protocol. Nucleic acid products were purified using Zymo Oligo Clean and Concentrator kit. The purified cDNA was then amplified via PCR (Phusion High-Fidelity PCR Master Mix with HF Buffer; Thermo Scientific™) using PCR primers RP635, RP672 resulting in 95 bp product as observed on an agarose gel. Next, the 95 bp DNA product was amplified using nested PCR primer RP635A and RP672A to increase length to 145 bp for downstream high-throughput sequencing. The 145 bp product was purified using Zymo Oligo Clean and Concentrator kit. Finally, the 145 bp PCR DNA products were submitted for next-generation high-throughput sequencing (Genewiz from Azenta Life Sciences). Reads were aligned to the 145 bp reference sequence with bowtie2 aligner, samtools were used to extract the tags from bam files representing distance from template, substitution, and gap opening (NM, XM,XO). All plots were generated from extracted counts using ggplot in R.

Nucleotide analogs: 2′-O-methyl-NTPs, N6-methyl-ATP, 5-methyl-CTP, and Pseudouridine-Triphosphate were purchased from Trilink Biotechnologies. 2′-F-NTPs were purchased from Jena Biosciences.

### Example 2: Mutant Polθ Proteins

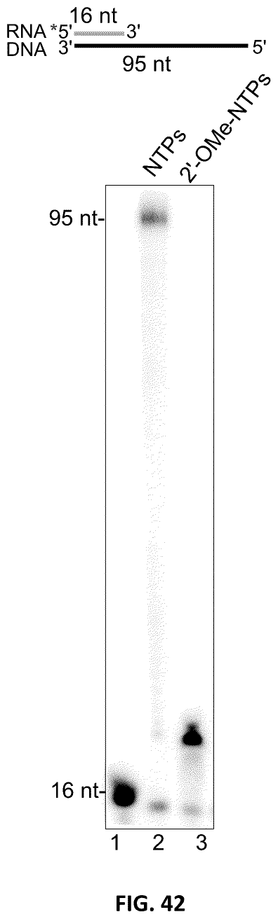

In an effort to improve the utility of the Polθ in large-scale production of RNA, a novel mutant of Polθ PolθDL, was developed. PolθDL, like other steric-gate Polθ proteins, is capable of producing full-length RNA molecules with canonical NTPs (FIG. 42), however PolθDL exhibits improved solubility in water. The improved solubility allows for DNA-dependent RNA synthesis at higher concentrations, allowing for reduced reaction volumes and associated time and labor.

### Example 3: Peptide and Nucleic Acid Sequences

Presented herein are the peptide sequences and the calculated nucleic acid sequences for the peptides. The amino acid sequences were calculated using EMBOSS Backtranambig—a program that reads a protein sequence and writes the nucleic acid sequence it could have come from. It does this by using nucleotide ambiguity codes that represent all possible codons for each amino acid (Table 1).

Polθ₁₇₉₂₋₂₅₉₀
Amino acid sequence:
(SEQ ID NO: 1)
GFKDNSPISDTSFSLQLSQDGLQLTPASSSSESLSIIDVASDQNLFQTFIKEWRCKK
RFSISLACEKIRSLTSSKTATIGSRFKQASSPQEIPIRDDGFPIKGCDDTLVVGLAVC
WGGRDAYYFSLQKEQKHSEISASLVPPSLDPSLTLKDRMWYLQSCLRKESDKEC
SVVIYDFIQSYKILLLSCGISLEQSYEDPKVACWLLDPDSQEPTLHSIVTSFLPHELP
LLEGMETSQGIQSLGLNAGSEHSGRYRASVESILIFNSMNQLNSLLQKENLQDVF
RKVEMPSQYCLALLELNGIGFSTAECESQKHIMQAKLDAIETQAYQLAGHSFSFT
SSDDIAEVLFLELKLPPNREMKNQGSKKTLGSTRRGIDNGRKLRLGRQFSTSKDV
LNKLKALHPLPGLILEWRRITNAITKVVFPLQREKCLNPFLGMERIYPVSQSHTAT
GRITFTEPNIQNVPRDFEIKMPTLVGESPPSQAVGKGLLPMGRGKYKKGFSVNPR
CQAQMEERAADRGMPFSISMRHAFVPFPGGSILAADYSQLELRILAHLSHDRRLI
QVLNTGADVFRSIAAEWKMIEPESVGDDLRQQAKQICYGIIYGMGAKSLGEQMG
IKENDAACYIDSFKSRYTGINQFMTETVKNCKRDGFVQTILGRRRYLPGIKDNNP
YRKAHAERQAINTIVQGSAADIVKIATVNIQKQLETFHSTFKSHGHREGMLQSDQ
TGLSRKRKLQGMFCPIRGGFFILQLHDELLYEVAEEDVVQVAQIVKNEMESAVK
LSVKLKVKVKIGASWGELKDFDV.
Nucleic acid sequence:
(SEQ ID NO: 2)
GGNTTYAARGAYAAYWSNCCNATHWSNGAYACNWSNTTYWSNYTNCARYTN
WSNCARGAYGGNYTNCARYTNACNCCNGCNWSNWSNWSNWSNGARWSNYT
NWSNATHATHGAYGTNGCNWSNGAYCARAAYYTNTTYCARACNTTYATHAA
RGARTGGMGNTGYAARAARMGNTTYWSNATHWSNYTNGCNTGYGARAARAT
HMGNWSNYTNACNWSNWSNAARACNGCNACNATHGGNWSNMGNTTYAARC
ARGCNWSNWSNCCNCARGARATHCCNATHMGNGAYGAYGGNTTYCCNATHA
ARGGNTGYGAYGAYACNYTNGTNGTNGGNYTNGCNGTNTGYTGGGGNGGNM
GNGAYGCNTAYTAYTTYWSNYTNCARAARGARCARAARCAYWSNGARATHW
SNGCNWSNYTNGTNCCNCCNWSNYTNGAYCCNWSNYTNACNYTNAARGAYM
GNATGTGGTAYYTNCARWSNTGYYTNMGNAARGARWSNGAYAARGARTGY
WSNGTNGTNATHTAYGAYTTYATHCARWSNTAYAARATHYTNYTNYTNWSN
TGYGGNATHWSNYTNGARCARWSNTAYGARGAYCCNAARGINGCNTGYTGG
YTNYTNGAYCCNGAYWSNCARGARCCNACNYTNCAYWSNATHGTNACNWSN
TTYYTNCCNCAYGARYTNCCNYTNYTNGARGGNATGGARACNWSNCARGGN
ATHCARWSNYTNGGNYTNAAYGCNGGNWSNGARCAYWSNGGNMGNTAYMG
NGCNWSNGTNGARWSNATHYTNATHTTYAAYWSNATGAAYCARYTNAAYWS
NYTNYTNCARAARGARAAYYTNCARGAYGTNTTYMGNAARGTNGARATGCC
NWSNCARTAYTGYYTNGCNYTNYTNGARYTNAAYGGNATHGGNTTYWSNAC
NGCNGARTGYGARWSNCARAARCAYATHATGCARGCNAARYTNGAYGCNAT
HGARACNCARGCNTAYCARYTNGCNGGNCAYWSNTTYWSNTTYACNWSNWS
NGAYGAYATHGCNGARGTNYTNTTYYTNGARYTNAARYTNCCNCCNAAYMG
NGARATGAARAAYCARGGNWSNAARAARACNYTNGGNWSNACNMGNMGNG
GNATHGAYAAYGGNMGNAARYTNMGNYTNGGNMGNCARTTYWSNACNWSN
AARGAYGTNYTNAAYAARYTNAARGCNYTNCAYCCNYTNCCNGGNYTNATH
YTNGARTGGMGNMGNATHACNAAYGCNATHACNAARGTNGTNTTYCCNYTN
CARMGNGARAARTGYYTNAAYCCNTTYYTNGGNATGGARMGNATHTAYCCN
GTNWSNCARWSNCAYACNGCNACNGGNMGNATHACNTTYACNGARCCNAA
YATHCARAAYGTNCCNMGNGAYTTYGARATHAARATGCCNACNYTNGTNGG
NGARWSNCCNCCNWSNCARGCNGTNGGNAARGGNYTNYTNCCNATGGGNM
GNGGNAARTAYAARAARGGNTTYWSNGTNAAYCCNMGNTGYCARGCNCARA
TGGARGARMGNGCNGCNGAYMGNGGNATGCCNTTYWSNATHWSNATGMGN
CAYGCNTTYGTNCCNTTYCCNGGNGGNWSNATHYTNGCNGCNGAYTAYWSN
CARYTNGARYTNMGNATHYTNGCNCAYYTNWSNCAYGAYMGNMGNYTNAT
HCARGTNYTNAAYACNGGNGCNGAYGTNTTYMGNWSNATHGCNGCNGARTG
GAARATGATHGARCCNGARWSNGTNGGNGAYGAYYTNMGNCARCARGCNA
ARCARATHTGYTAYGGNATHATHTAYGGNATGGGNGCNAARWSNYTNGGNG
ARCARATGGGNATHAARGARAAYGAYGCNGCNTGYTAYATHGAYWSNTTYA
ARWSNMGNTAYACNGGNATHAAYCARTTYATGACNGARACNGTNAARAAYT
GYAARMGNGAYGGNTTYGTNCARACNATHYTNGGNMGNMGNMGNTAYYTN
CCNGGNATHAARGAYAAYAAYCCNTAYMGNAARGENCAYGCNGARMGNCA
RGCNATHAAYACNATHGTNCARGGNWSNGCNGCNGAYATHGTNAARATHGC
NACNGTNAAYATHCARAARCARYTNGARACNTTYCAYWSNACNTTYAARWS
NCAYGGNCAYMGNGARGGNATGYTNCARWSNGAYCARACNGGNYTNWSNM
GNAARMGNAARYTNCARGGNATGTTYTGYCCNATHMGNGGNGGNTTYTTYA
THYTNCARYTNCAYGAYGARYTNYTNTAYGARGTNGCNGARGARGAYGING
TNCARGTNGCNCARATHGTNAARAAYGARATGGARWSNGCNGTNAARYTNW
SNGTNAARYTNAARGTNAARGTNAARATHGGNGCNWSNTGGGGNGARYTNA
ARGAYTTYGAYGTN.
Full Length Human Pole
Amino acid sequence:
(SEQ ID NO: 17)
MNLLRRSGKRRRSESGSDSFSGSGGDSSASPQFLSGSVLSPPPGLGRCLKAAAAG
ECKPTVPDYERDKLLLANWGLPKAVLEKYHSFGVKKMFEWQAECLLLGQVLEG
KNLVYSAPTSAGKTLVAELLILKRVLEMRKKALFILPFVSVAKEKKYYLQSLFQE
VGIKVDGYMGSTSPSRHFSSLDIAVCTIERANGLINRLIEENKMDLLGMVVVDEL
HMLGDSHRGYLLELLLTKICYITRKSASCQADLASSLSNAVQIVGMSATLPNLEL
VASWLNAELYHTDFRPVPLLESVKVGNSIYDSSMKLVREFEPMLQVKGDEDHV
VSLCYETICDNHSVLLFCPSKKWCEKLADIIAREFYNLHHQAEGLVKPSECPPVIL
EQKELLEVMDQLRRLPSGLDSVLQKTVPWGVAFHHAGLTFEERDIIEGAFRQGLI
RVLAATSTLSSGVNLPARRVIIRTPIFGGRPLDILTYKQMVGRAGRKGVDTVGESI
LICKNSEKSKGIALLQGSLKPVRSCLQRREGEEVTGSMIRAILEIIVGGVASTSQD
MHTYAACTFLAASMKEGKQGIQRNQESVQLGAIEACVMWLLENEFIQSTEASDG
TEGKVYHPTHLGSATLSSSLSPADTLDIFADLQRAMKGFVLENDLHILYLVTPMF
EDWTTIDWYRFFCLWEKLPTSMKRVAELVGVEEGFLARCVKGKVVARTERQHR
QMAIHKRFFTSLVLLDLISEVPLREINQKYGCNRGQIQSLQQSAAVYAGMITVFS
NRLGWHNMELLLSQFQKRLTFGIQRELCDLVRVSLLNAQRARVLYASGFHTVA
DLARANIVEVEVILKNAVPFKSARKAVDEEEEAVEERRNMRTIWVTGRKGLTER
EAAALIVEEARMILQQDLVEMGVQWNPCALLHSSTCSLTHSESEVKEHTFISQTK
SSYKKLTSKNKSNTIFSDSYIKHSPNIVQDLNKSREHTSSFNCNFQNGNQEHQTCS
IFRARKRASLDINKEKPGASQNEGKTSDKKVVQTFSQKTKKAPLNFNSEKMSRSF
RSWKRRKHLKRSRDSSPLKDSGACRIHLQGQTLSNPSLCEDPFTLDEKKTEFRNS
GPFAKNVSLSGKEKDNKTSFPLQIKQNCSWNITLTNDNFVEHIVTGSQSKNVTCQ
ATSVVSEKGRGVAVEAEKINEVLIQNGSKNQNVYMKHHDIHPINQYLRKQSHEQ
TSTITKQKNIIERQMPCEAVSSYINRDSNVTINCERIKLNTEENKPSHFQALGDDIS
RTVIPSEVLPSAGAFSKSEGQHENFLNISRLQEKTGTYTTNKTKNNHVSDLGLVL
CDFEDSFYLDTQSEKIIQQMATENAKLGAKDTNLAAGIMQKSLVQQNSMNSFQK
ECHIPFPAEQHPLGATKIDHLDLKTVGTMKQSSDSHGVDILTPESPIFHSPILLEEN
GLFLKKNEVSVTDSQLNSFLQGYQTQETVKPVILLIPQKRTPTGVEGECLPVPETS
LNMSDSLLFDSFSDDYLVKEQLPDMQMKEPLPSEVTSNHFSDSLCLQEDLIKKSN
VNENQDTHQQLTCSNDESIIFSEMDSVQMVEALDNVDIFPVQEKNHTVVSPRALE
LSDPVLDEHHQGDQDGGDQDERAEKSKLTGTRQNHSFIWSGASFDLSPGLQRIL
DKVSSPLENEKLKSMTINFSSLNRKNTELNEEQEVISNLETKQVQGISFSSNNEVK
SKIEMLENNANHDETSSLLPRKESNIVDDNGLIPPTPIPTSASKLTFPGILETPVNP
WKTNNVLQPGESYLFGSPSDIKNHDLSPGSRNGFKDNSPISDTSFSLQLSQDGLQL
TPASSSSESLSIIDVASDQNLFQTFIKEWRCKKRFSISLACEKIRSLTSSKTATIGSRF
KQASSPQEIPIRDDGFPIKGCDDTLVVGLAVCWGGRDAYYFSLQKEQKHSEISAS
LVPPSLDPSLTLKDRMWYLQSCLRKESDKECSVVIYDFIQSYKILLLSCGISLEQS
YEDPKVACWLLDPDSQEPTLHSIVTSFLPHELPLLEGMETSQGIQSLGLNAGSEHS
GRYRASVESILIFNSMNQLNSLLQKENLQDVFRKVEMPSQYCLALLELNGIGFST
AECESQKHIMQAKLDAIETQAYQLAGHSFSFTSSDDIAEVLFLELKLPPNREMKN
QGSKKTLGSTRRGIDNGRKLRLGRQFSTSKDVLNKLKALHPLPGLILEWRRITNAI
TKVVFPLQREKCLNPFLGMERIYPVSQSHTATGRITFTEPNIQNVPRDFEIKMPTL
VGESPPSQAVGKGLLPMGRGKYKKGFSVNPRCQAQMEERAADRGMPFSISMRH
AFVPFPGGSILAADYSQLELRILAHLSHDRRLIQVLNTGADVFRSIAAEWKMIEPE
SVGDDLRQQAKQICYGIIYGMGAKSLGEQMGIKENDAACYIDSFKSRYTGINQF
MTETVKNCKRDGFVQTILGRRRYLPGIKDNNPYRKAHAERQAINTIVQGSAADIV
KIATVNIQKQLETFHSTFKSHGHREGMLQSDQTGLSRKRKLQGMFCPIRGGFFIL
QLHDELLYEVAEEDVVQVAQIVKNEMESAVKLSVKLKVKVKIGASWGELKDFD
V
PolθRP1
Amino acid sequence:
(SEQ ID NO: 18)
MNLLRRSGKRRRSESGSDSFSGSGGDSSASPQFLSGSVLSPPPGLGRCLKAAAAG
ECKPTVPDYERDKLLLANWGLPKAVLEKYHSFGVKKMFEWQAECLLLGQVLEG
KNLVYSAPTSAGKTLVAELLILKRVLEMRKKALFILPFVSVAKEKKYYLQSLFQE
VGIKVDGYMGSTSPSRHFSSLDIAVCTIERANGLINRLIEENKMDLLGMVVVDEL
HMLGDSHRGYLLELLLTKICYITRKSASCQADLASSLSNAVQIVGMSATLPNLEL
VASWLNAELYHTDFRPVPLLESVKVGNSIYDSSMKLVREFEPMLQVKGDEDHV
VSLCYETICDNHSVLLFCPSKKWCEKLADIIAREFYNLHHQAEGLVKPSECPPVIL
EQKELLEVMDQLRRLPSGLDSVLQKTVPWGVAFHHAGLTFEERDIIEGAFRQGLI
RVLAATSTLSSGVNLPARRVIIRTPIFGGRPLDILTYKQMVGRAGRKGVDTVGESI
LICKNSEKSKGIALLQGSLKPVRSCLQRREGEEVTGSMIRAILEIIVGGVASTSQD
MHTYAACTFLAASMKEGKQGIQRNQESVQLGAIEACVMWLLENEFIQSTEASDG
TEGKVYHPTHLGSATLSSSLSPADTLDIFADLQRAMKGFVLENDLHILYLVTPMF
EDWTTIDWYRFFCLWEKLPTSMKRVAELVGVEEGFLARCVKGKVVARTERQHR
QMAIHKRFFTSLVLLDLISEVPLREINQKYGCNRGQIQSLQQSAAVYAGMITVFS
NRLGWHNMELLLSQFQKRLTFGIQRELCDLVRVSLLNAQRARVLYASGFHTVA
DLARANIVEVEVILKNAVPFKSARKAVDEEEEAVEERRNMRTIWVTGRKGLTER
EAAALIVEEARMILQQDLVEMGVQWNPCALLHSSTCSLTHSESEVKEHTFISQTK
SSYKKLTSKNKSNTIFSDSYIKHSPNIVQDLNKSREHTSSFNCNFQNGNQEHQTCS
IFRARKRASLDINKEKPGASQNEGKTSDKKVVQTFSQKTKKAPLNFNSEKMSRSF
RSWKRRKHLKRSRDSSPLKDSGACRIHLQGQTLSNPSLCEDPFTLDEKKTEFRNS
GPFAKNVSLSGKEKDNKTSFPLQIKQNCSWNITLTNDNFVEHIVTGSQSKNVTCQ
ATSVVSEKGRGVAVEAEKINEVLIQNGSKNQNVYMKHHDIHPINQYLRKQSHEQ
TSTITKQKNIIERQMPCEAVSSYINRDSNVTINCERIKLNTEENKPSHFQALGDDIS
RTVIPSEVLPSAGAFSKSEGQHENFLNISRLQEKTGTYTTNKTKNNHVSDLGLVL
CDFEDSFYLDTQSEKIIQQMATENAKLGAKDTNLAAGIMQKSLVQQNSMNSFQK
ECHIPFPAEQHPLGATKIDHLDLKTVGTMKQSSDSHGVDILTPESPIFHSPILLEEN
GLFLKKNEVSVTDSQLNSFLQGYQTQETVKPVILLIPQKRTPTGVEGECLPVPETS
LNMSDSLLFDSFSDDYLVKEQLPDMQMKEPLPSEVTSNHFSDSLCLQEDLIKKSN
VNENQDTHQQLTCSNDESIIFSEMDSVQMVEALDNVDIFPVQEKNHTVVSPRALE
LSDPVLDEHHQGDQDGGDQDERAEKSKLTGTRQNHSFIWSGASFDLSPGLQRIL
DKVSSPLENEKLKSMTINFSSLNRKNTELNEEQEVISNLETKQVQGISFSSNNEVK
SKIEMLENNANHDETSSLLPRKESNIVDDNGLIPPTPIPTSASKLTFPGILETPVNP
WKTNNVLQPGESYLFGSPSDIKNHDLSPGSRNGFKDNSPISDTSFSLQLSQDGLQL
TPASSSSESLSIIDVASDQNLFQTFIKEWRCKKRFSISLACEKIRSLTSSKTATIGSRF
KQASSPQEIPIRDDGFPIKGCDDTLVVGLAVCWGGRDAYYFSLQKEQKHSEISAS
LVPPSLDPSLTLKDRMWYLQSCLRKESDKECSVVIYDFIQSYKILLLSCGISLEQS
YEDPKVACWLLDPDSQEPTLHSIVTSFLPHELPLLEGMETSQGIQSLGLNAGSEHS
GRYRASVESILIFNSMNQLNSLLQKENLQDVFRKVEMPSQYCLALLELNGIGFST
AECESQKHIMQAKLDAIETQAYQLAGHSFSFTSSDDIAEVLFLELKLPPNREMKN
QGSKKTLGSTRRGIDNGRKLRLGRQFSTSKDVLNKLKALHPLPGLILEWRRITNAI
TKVVFPLQREKCLNPFLGMERIYPVSQSHTATGRITFTEPNIQNVPRDFEIKMPTL
VGESPPSQAVGKGLLPMGRGKYKKGFSVNPRCQAQMEERAADRGMPFSISMRH
AFVPFPGGSILAADYSQLGLRILAHLSHDRRLIQVLNTGADVFRSIAAEWKMIEPE
SVGDDLRQQAKQICYGIIYGMGAKSLGEQMGIKENDAACYIDSFKSRYTGINQF
MTETVKNCKRDGFVQTILGRRRYLPGIKDNNPYRKAHAERQAINTIVQGSAADIV
KIATVNIQKQLETFHSTFKSHGHREGMLQSDQTGLSRKRKLQGMFCPIRGGFFIL
QLHDELLYEVAEEDVVQVAQIVKNEMESAVKLSVKLKVKVKIGASWGELKDFD
V
PolθRP2
Amino acid sequence:
(SEQ ID NO: 19)
MNLLRRSGKRRRSESGSDSFSGSGGDSSASPQFLSGSVLSPPPGLGRCLKAAAAG
ECKPTVPDYERDKLLLANWGLPKAVLEKYHSFGVKKMFEWQAECLLLGQVLEG
KNLVYSAPTSAGKTLVAELLILKRVLEMRKKALFILPFVSVAKEKKYYLQSLFQE
VGIKVDGYMGSTSPSRHFSSLDIAVCTIERANGLINRLIEENKMDLLGMVVVDEL
HMLGDSHRGYLLELLLTKICYITRKSASCQADLASSLSNAVQIVGMSATLPNLEL
VASWLNAELYHTDFRPVPLLESVKVGNSIYDSSMKLVREFEPMLQVKGDEDHV
VSLCYETICDNHSVLLFCPSKKWCEKLADIIAREFYNLHHQAEGLVKPSECPPVIL
EQKELLEVMDQLRRLPSGLDSVLQKTVPWGVAFHHAGLTFEERDIIEGAFRQGLI
RVLAATSTLSSGVNLPARRVIIRTPIFGGRPLDILTYKQMVGRAGRKGVDTVGESI
LICKNSEKSKGIALLQGSLKPVRSCLQRREGEEVTGSMIRAILEIIVGGVASTSQD
MHTYAACTFLAASMKEGKQGIQRNQESVQLGAIEACVMWLLENEFIQSTEASDG
TEGKVYHPTHLGSATLSSSLSPADTLDIFADLQRAMKGFVLENDLHILYLVTPMF
EDWTTIDWYRFFCLWEKLPTSMKRVAELVGVEEGFLARCVKGKVVARTERQHR
QMAIHKRFFTSLVLLDLISEVPLREINQKYGCNRGQIQSLQQSAAVYAGMITVFS
NRLGWHNMELLLSQFQKRLTFGIQRELCDLVRVSLLNAQRARVLYASGFHTVA
DLARANIVEVEVILKNAVPFKSARKAVDEEEEAVEERRNMRTIWVTGRKGLTER
EAAALIVEEARMILQQDLVEMGVQWNPCALLHSSTCSLTHSESEVKEHTFISQTK
SSYKKLTSKNKSNTIFSDSYIKHSPNIVQDLNKSREHTSSFNCNFQNGNQEHQTCS
IFRARKRASLDINKEKPGASQNEGKTSDKKVVQTFSQKTKKAPLNFNSEKMSRSF
RSWKRRKHLKRSRDSSPLKDSGACRIHLQGQTLSNPSLCEDPFTLDEKKTEFRNS
GPFAKNVSLSGKEKDNKTSFPLQIKQNCSWNITLTNDNFVEHIVTGSQSKNVTCQ
ATSVVSEKGRGVAVEAEKINEVLIQNGSKNQNVYMKHHDIHPINQYLRKQSHEQ
TSTITKQKNIIERQMPCEAVSSYINRDSNVTINCERIKLNTEENKPSHFQALGDDIS
RTVIPSEVLPSAGAFSKSEGQHENFLNISRLQEKTGTYTTNKTKNNHVSDLGLVL
CDFEDSFYLDTQSEKIIQQMATENAKLGAKDTNLAAGIMQKSLVQQNSMNSFQK
ECHIPFPAEQHPLGATKIDHLDLKTVGTMKQSSDSHGVDILTPESPIFHSPILLEEN
GLFLKKNEVSVTDSQLNSFLQGYQTQETVKPVILLIPQKRTPTGVEGECLPVPETS
LNMSDSLLFDSFSDDYLVKEQLPDMQMKEPLPSEVTSNHFSDSLCLQEDLIKKSN
VNENQDTHQQLTCSNDESIIFSEMDSVQMVEALDNVDIFPVQEKNHTVVSPRALE
LSDPVLDEHHQGDQDGGDQDERAEKSKLTGTRQNHSFIWSGASFDLSPGLQRIL
DKVSSPLENEKLKSMTINFSSLNRKNTELNEEQEVISNLETKQVQGISFSSNNEVK
SKIEMLENNANHDETSSLLPRKESNIVDDNGLIPPTPIPTSASKLTFPGILETPVNP
WKTNNVLQPGESYLFGSPSDIKNHDLSPGSRNGFKDNSPISDTSFSLQLSQDGLQL
TPASSSSESLSIIDVASDQNLFQTFIKEWRCKKRFSISLACEKIRSLTSSKTATIGSRF
KQASSPQEIPIRDDGFPIKGCDDTLVVGLAVCWGGRDAYYFSLQKEQKHSEISAS
LVPPSLDPSLTLKDRMWYLQSCLRKESDKECSVVIYDFIQSYKILLLSCGISLEQS
YEDPKVACWLLDPDSQEPTLHSIVTSFLPHELPLLEGMETSQGIQSLGLNAGSEHS
GRYRASVESILIFNSMNQLNSLLQKENLQDVFRKVEMPSQYCLALLELNGIGFST
AECESQKHIMQAKLDAIETQAYQLAGHSFSFTSSDDIAEVLFLELKLPPNREMKN
QGSKKTLGSTRRGIDNGRKLRLGRQFSTSKDVLNKLKALHPLPGLILEWRRITNAI
TKVVFPLQREKCLNPFLGMERIYPVSQSHTATGRITFTEPNIQNVPRDFEIKMPTL
VGESPPSQAVGKGLLPMGRGKYKKGFSVNPRCQAQMEERAADRGMPFSISMRH
AFVPFPGGSFLAADYSQLGLRILAHLSHDRRLIQVLNTGADVFRSIAAEWKMIEP
ESVGDDLRQQAKQICYGIIYGMGAKSLGEQMGIKENDAACYIDSFKSRYTGINQF
MTETVKNCKRDGFVQTILGRRRYLPGIKDNNPYRKAHAERQAINTIVQGSAADIV
KIATVNIQKQLETFHSTFKSHGHREGMLQSDQTGLSRKRKLQGMFCPIRGGFFIL
QLHDELLYEVAEEDVVQVAQIVKNEMESAVKLSVKLKVKVKIGASWGELKDFD
V
Polθ₁₈₁₉₋₂₅₉₀
Amino acid sequence:
(SEQ ID NO: 20)
SSSSESLSIIDVASDQNLFQTFIKEWRCKKRFSISLACEKIRSLTSSKTATIGSRFKQ
ASSPQEIPIRDDGFPIKGCDDTLVVGLAVCWGGRDAYYFSLQKEQKHSEISASLV
PPSLDPSLTLKDRMWYLQSCLRKESDKECSVVIYDFIQSYKILLLSCGISLEQSYE
DPKVACWLLDPDSQEPTLHSIVTSFLPHELPLLEGMETSQGIQSLGLNAGSEHSGR
YRASVESILIFNSMNQLNSLLQKENLQDVFRKVEMPSQYCLALLELNGIGFSTAE
CESQKHIMQAKLDAIETQAYQLAGHSFSFTSSDDIAEVLFLELKLPPNREMKNQG
SKKTLGSTRRGIDNGRKLRLGRQFSTSKDVLNKLKALHPLPGLILEWRRITNAITK
VVFPLQREKCLNPFLGMERIYPVSQSHTATGRITFTEPNIQNVPRDFEIKMPTLVG
ESPPSQAVGKGLLPMGRGKYKKGFSVNPRCQAQMEERAADRGMPFSISMRHAF
VPFPGGSILAADYSQLELRILAHLSHDRRLIQVLNTGADVFRSIAAEWKMIEPESV
GDDLRQQAKQICYGIIYGMGAKSLGEQMGIKENDAACYIDSFKSRYTGINQFMT
ETVKNCKRDGFVQTILGRRRYLPGIKDNNPYRKAHAERQAINTIVQGSAADIVKI
ATVNIQKQLETFHSTFKSHGHREGMLQSDQTGLSRKRKLQGMFCPIRGGFFILQL
HDELLYEVAEEDVVQVAQIVKNEMESAVKLSVKLKVKVKIGASWGELKDFDV.
PolθDL Steric Gate
Amino acid sequence:
(SEQ ID NO: 21)
SSSSESLSIIDVASDQNLFQTFIKEWRCKKRFSISLACEKIRGSGDDTLVVGLAVCW
GGRDAYYFSLGGSGGLDPSLTLKDRMWYLQSCLRKESDKECSVVIYDFIQSYKIL
LLSCGISLEQSYEDPKVACWLLDPDSQEPTLHSIVTSFLPHELPLLEGMETSQGIQS
LGLNAGSEHSGRYRASVESILIFNSMNQLNSLLQKENLQDVFRKVEMPSQYCLAL
LELNGIGFSTAECESQKHIMQAKLDAIETQAYQLAGHSFSFTSSDDIAEVLFLELKL
PPGGSGGQFSTSKDVLNKLKALHPLPGLILEWRRITNAITKVVFPLQREKCLNPFL
GMERIYPVSQSHTATGRITFTEPNIQNVPRDFEIKMGGSGGMPFSISMRHAFVPFP
GGSILAADYSQLGLRILAHLSHDRRLIQVLNTGADVFRSIAAEWKMIEPESVGDD
LRQQAKQICYGIIYGMGAKSLGEQMGIKENDAACYIDSFKSRYTGINQFMTETVK
NCKRDGFVQTILGRRRYLPGIKDNNPYRKAHAERQAINTIVQGSAADIVKIATVNI
QKQLETFHSTFKSHGHREGMLQSDGGSGGCPIRGGFFILQLHDELLYEVAEEDVV
QVAQIVKNEMESAVKLSVKLKVKVKIGASWGELKDFDV.

TABLE 1
Nucleic acid code to generate computed sequences

| Code | Meaning | Etymology | Complement | Opposite |
|---|---|---|---|---|
| A | A | Adenosine | T | B |
| T/U | T or U | Thymidine/Uridine | A | V |
| G | G | Guanine | C | H |
| C | C | Cytidine | G | D |
| K | G or T | Keto | M | M |
| M | A or C | Amino | K | K |
| R | A or G | Purine | Y | Y |
| Y | C or T | Pyrimidine | R | R |
| S | C or G | Strong | S | W |
| W | A or T | Weak | W | S |
| B | C or G | not A (B comes | V | A |
| | or T | after A) | | |
| V | A or C | not T/U (V comes | B | T/U |
| | or G | after U) | | |
| H | A or C | not G (H comes | D | G |
| | or T | after G) | | |
| D | A or G | not C (D comes | H | C |
| | or T | after C) | | |
| X/N | G or A or | any | N | . |
| | T or C | | | |
| . | not G or | | . | N |
| | A or T | | | |
| | or C | | | |
| — | gap of | | | |
| | indeterminate | | | |
| | length | | | |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of synthesizing a sequence-specific oligonucleotide, the method comprising the steps of:
providing a DNA template in a solution;
contacting the nucleic acid template in the solution with a primer; and
contacting the nucleic acid primer-template in the solution with an A-family DNA polymerase mutant or variant thereof.

2. The method of claim 1, wherein the sequence-specific oligonucleotide comprises DNA, RNA, or a combination thereof.

3. The method of claim 1, wherein the solution comprises at least one selected from the group consisting of divalent cations, nucleotide triphosphates (NTPs), deoxynucleotide triphosphates (dNTPs), chemically modified NTPs, chemically modified dNTPs, chemically modified cytidine, chemically modified uridine, chemically modified guanosine, chemically modified adenosine, non-canonical NTPs, and non-canonical dNTPs.

4. The method of claim 3, wherein the solution comprises chemically modified NTPs wherein the NTPs are chemically modified, wherein the chemical modification comprises at least one selected from the group consisting of ribose modifications, base modifications, phosphate modifications, alpha-phosphate modification, alpha-thiophosphate modifications, 3′-ribose, and 2′-ribose modifications.

5. The method of claim 1, wherein the primer comprises at least one selected from the group consisting of: an RNA, a chemically modified RNA, a DNA, a chemically modified DNA, a DNA-RNA chimera, a chemically modified DNA-RNA chimera, and a DNA, RNA, or DNA-RNA chimera with a non-complementary 5′-single-strand overhang.

6. The method of claim 1, wherein the primer is complementary or partially complementary with the template, wherein a partially complementary primer has a non-complementary 5′-overhang or has a portion of bases that are not complementary with the template

7. The method of claim 1, wherein the primer comprises a chemically modified RNA wherein the chemical modification comprises at least one selected from the group consisting of a base modification, a ribose modification, a phosphate modification, a 2′-ribose modification, and a 3′-ribose modification.

8. The method of claim 1, wherein the primer comprises a chemically modified DNA wherein the chemical modification comprises at least one selected from the group consisting of a base modification, a deoxyribose modification, a phosphate modification, a 2′-deoxyribose modification, and a 3′-deoxyribose modification.

9. The method of claim 1, wherein the primer comprises a chemically modified DNA-RNA chimera, wherein the chemical modification comprises at least one selected from the group consisting of a base modification, a ribose modification, a deoxyribose modification, a phosphate modification, a 2′-ribose modification, a 2′-deoxyribose modification, a 3′-ribose modification, and a 3′-deoxyribose modification.

10. The method of claim 1, where the A-family DNA polymerase mutant or variant thereof comprises a DNA polymerase theta (Pol) mutant.

11. The method of claim 10, wherein the DNA polymerase theta (Polθ) mutant comprises the amino acid sequence of SEQ ID NO: 1 or a variant thereof.

12. The method of claim 11, wherein the DNA polymerase theta (Polθ) mutant comprises at least one mutation relative to SEQ ID NO:1;
wherein at least one mutation is E544X or E544G; and
wherein X is any proteogenic amino acid.

13. The method of claim 12, wherein the DNA polymerase theta (Polθ) mutant further comprises at least one mutation selected from the group consisting of I535X and I535F;
wherein X is any proteogenic amino acid.

14. The method of claim 10, wherein the DNA polymerase theta (Polθ) mutant comprises the amino acid sequence of SEQ ID NO: 17 or a variant thereof.

15. The method of claim 14, wherein the DNA polymerase theta (Polθ) mutant comprises at least one mutation relative to SEQ ID NO:17;
wherein at least one mutation is E2335X or E2335G; and
wherein X is any proteogenic amino acid.

16. The method of claim 15, wherein the DNA polymerase theta (Polθ) mutant further comprises at least one mutation selected from the group consisting of I2326X and I2326F;
wherein X is any proteogenic amino acid.

17. The method of claim 10, wherein the DNA polymerase theta (Polθ) mutant comprises the amino acid sequence of SEQ ID NO:21 or a variant thereof.

18. A kit for preparing an oligonucleotide comprising a DNA polymerase theta (Polθ) mutant comprising an amino acid sequence selected from the group consisting of: SEQ ID NO: 1 or a variant thereof, SEQ ID NO: 17 or a variant thereof, and SEQ ID NO:21 or a variant thereof.

19. The kit of claim 18, wherein the DNA polymerase theta (Polθ) mutant comprises the amino acid sequence of SEQ ID NO:1;
wherein the mutant comprises at least one mutation relative to SEQ ID NO: 1;
wherein at least one mutation is E544X or E544G; and
wherein X is any proteogenic amino acid.

20. The kit of claim 19, wherein the DNA polymerase theta (Polθ) mutant further comprises at least one mutation selected from the group consisting of I535X or I535F;
wherein X is any proteogenic amino acid.

2. The kit of claim 18, wherein the DNA polymerase theta (Polθ) mutant comprises the amino acid sequence of SEQ ID NO:17;
wherein the mutant comprises at least one mutation relative to SEQ ID NO: 17;
wherein at least one mutation is E2335X or E2335G; and
wherein X is any proteogenic amino acid.

22. The kit of claim 21, wherein the DNA polymerase theta (Polθ) mutant further comprises at least one mutation selected from the group consisting of I2326X and I2326F;
wherein X is any proteogenic amino acid.

23. The kit of claim 18, wherein the kit further comprises one or more solutions comprising one or more selected from the group consisting of: a divalent cation, NTPs, dNTPs, chemically modified NTPs, chemically modified dNTPs, Tris hydrochloride, glycerol, NP-40, bovine serum albumin (BSA), sodium chloride (NaCl), 1,4-dithiothreitol (DTT), and instructional material.

24. A DNA polymerase theta (Polθ) mutant comprising the amino acid sequence of SEQ ID NO:1;
wherein the mutant comprises at least one mutation;
wherein at least one mutation is E544X or E544G; and
wherein X is any proteogenic amino acid.

25. The DNA polymerase theta (Polθ) mutant of claim 24, wherein the mutant further comprises a mutation selected from the group consisting of I535X and I535F;
wherein X is any proteogenic amino acid.

26. A DNA polymerase theta (Polθ) mutant comprising the amino acid sequence of SEQ ID NO:17;
wherein the mutant comprises at least two mutations;
wherein a first mutation is E2335X or E2335G;
wherein a second mutation is I2326X or I2326F; and
wherein each instance of X is independently any proteogenic amino acid.

27. A DNA polymerase theta (Polθ) mutant comprising the amino acid sequence of SEQ ID NO:21.

28. A composition comprising the DNA polymerase theta (Polθ) mutant of claim 24 or a nucleic acid encoding the DNA polymerase theta (Polθ) mutant.

29. A composition comprising the DNA polymerase theta (Polθ) mutant of claim 25 or a nucleic acid encoding the DNA polymerase theta (Polθ) mutant.

30. A composition comprising the DNA polymerase theta (Polθ) mutant of claim 26 or a nucleic acid encoding the DNA polymerase theta (Polθ) mutant.

31. A composition comprising the DNA polymerase theta (Polθ) mutant of claim 27 or a nucleic acid encoding the DNA polymerase theta (Polθ) mutant.


SEQUENCE LISTING (21 sequences)

SEQ ID NO: 1 - protein, 799 aa, synthetic construct
GFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 2 - DNA, 2397 bp, synthetic construct
  source 1..2397: mol_type = other DNA
ggnttyaargayaaywsnccnathwsngayacnwsnttywsnytncarytnwsncargayggnytncarytnacnccngcnwsnwsnwsnwsngarwsnytnwsnathathgaygtngcnwsngaycaraayytnttycaracnttyathaargartggmgntgyaaraarmgnttywsnathwsnytngcntgygaraarathmgnwsnytnacnwsnwsnaaracngcnacnathggnwsnmgnttyaarcargcnwsnwsnccncargarathccnathmgngaygayggnttyccnathaarggntgygaygayacnytngtngtnggnytngcngtntgytggggnggnmgngaygcntaytayttywsnytncaraargarcaraarcaywsngarathwsngcnwsnytngtnccnccnwsnytngayccnwsnytnacnytnaargaymgnatgtggtayytncarwsntgyytnmgnaargarwsngayaargartgywsngtngtnathtaygayttyathcarwsntayaarathytnytnytnwsntgyggnathwsnytngarcarwsntaygargayccnaargtngcntgytggytnytngayccngaywsncargarccnacnytncaywsnathgtnacnwsnttyytnccncaygarytnccnytnytngarggnatggaracnwsncarggnathcarwsnytnggnytnaaygcnggnwsngarcaywsnggnmgntaymgngcnwsngtngarwsnathytnathttyaaywsnatgaaycarytnaaywsnytnytncaraargaraayytncargaygtnttymgnaargtngaratgccnwsncartaytgyytngcnytnytngarytnaayggnathggnttywsnacngcngartgygarwsncaraarcayathatgcargcnaarytngaygcnathgaracncargcntaycarytngcnggncaywsnttywsnttyacnwsnwsngaygayathgcngargtnytnttyytngarytnaarytnccnccnaaymgngaratgaaraaycarggnwsnaaraaracnytnggnwsnacnmgnmgnggnathgayaayggnmgnaarytnmgnytnggnmgncarttywsnacnwsnaargaygtnytnaayaarytnaargcnytncayccnytnccnggnytnathytngartggmgnmgnathacnaaygcnathacnaargtngtnttyccnytncarmgngaraartgyytnaayccnttyytnggnatggarmgnathtayccngtnwsncarwsncayacngcnacnggnmgnathacnttyacngarccnaayathcaraaygtnccnmgngayttygarathaaratgccnacnytngtnggngarwsnccnccnwsncargcngtnggnaarggnytnytnccnatgggnmgnggnaartayaaraarggnttywsngtnaayccnmgntgycargcncaratggargarmgngcngcngaymgnggnatgccnttywsnathwsnatgmgncaygcnttygtnccnttyccnggnggnwsnathytngcngcngaytaywsncarytngarytnmgnathytngcncayytnwsncaygaymgnmgnytnathcargtnytnaayacnggngcngaygtnttymgnwsnathgcngcngartggaaratgathgarccngarwsngtnggngaygayytnmgncarcargcnaarcarathtgytayggnathathtayggnatgggngcnaarwsnytnggngarcaratgggnathaargaraaygaygcngcntgytayathgaywsnttyaarwsnmgntayacnggnathaaycarttyatgacngaracngtnaaraaytgyaarmgngayggnttygtncaracnathytnggnmgnmgnmgntayytnccnggnathaargayaayaayccntaymgnaargcncaygcngarmgncargcnathaayacnathgtncarggnwsngcngcngayathgtnaarathgcnacngtnaayathcaraarcarytngaracnttycaywsnacnttyaarwsncayggncaymgngarggnatgytncarwsngaycaracnggnytnwsnmgnaarmgnaarytncarggnatgttytgyccnathmgnggnggnttyttyathytncarytncaygaygarytnytntaygargtngcngargargaygtngtncargtngcncarathgtnaaraaygaratggarwsngcngtnaarytnwsngtnaarytnaargtnaargtnaarathggngcnwsntggggngarytnaargayttygaygtn

SEQ ID NO: 3 - DNA, 16 bp, synthetic construct
  source 1..16: mol_type = other DNA
gcggagggcgataacg

SEQ ID NO: 4 - RNA, 16 nt, synthetic construct
  source 1..16: mol_type = other RNA
gcggagggcgataacg

SEQ ID NO: 5 - DNA, 95 bp, synthetic construct
  source 1..95: mol_type = other DNA
agactccgtatcgtaaagtgaccgacggtgttgtaactgacgaaattcactacctgtctgctatcgaagaaggcaactacgttatcgccctccgc

SEQ ID NO: 6 - DNA, 95 bp, synthetic construct; identical to SEQ ID NO: 5
  source 1..95: mol_type = other DNA
  modified_base 1: mod_base = OTHER; note = Conjugated to biotin

SEQ ID NO: 7 - DNA, 136 bp, synthetic construct
  source 1..136: mol_type = other DNA
tcctggccaatgagatggcagctgccaatggctgggcacacagactccgtatcgtaaagtgaccgacggtgttgtaactgacgaaattcactacctgtctgctatcgaagaaggcaactacgttatcgccctccgc

SEQ ID NO: 8 - DNA, 25 bp, synthetic construct
  source 1..25: mol_type = other DNA
aggcaaccgcgttatcgccctccgc

SEQ ID NO: 9 - protein, 25 aa, synthetic construct
AGGCAACGTCGTTATCGCCCTCCGC

SEQ ID NO: 10 - DNA, 25 bp, synthetic construct
  source 1..25: mol_type = other DNA
aggcaacgccgttatcgccctccgc

SEQ ID NO: 11 - DNA, 121 bp, synthetic construct
  source 1..121: mol_type = other DNA
gatcgattaatacgactcactatagggcggagggcgataacgtagttgccttcttcgatagcagacaggtagtgaatttcgtcagttacaacaccgtcggtcactttacgatacggagtct

SEQ ID NO: 12 - DNA, 121 bp, synthetic construct
  source 1..121: mol_type = other DNA
agactccgtatcgtaaagtgaccgacggtgttgtaactgacgaaattcactacctgtctgctatcgaagaaggcaactacgttatcgccctccgccctatagtgagtcgtattaatcgatc

SEQ ID NO: 13 - DNA, 23 bp, synthetic construct
  source 1..23: mol_type = other DNA
tcgagcacgttatcgccctccgc

SEQ ID NO: 14 - DNA, 200 bp, synthetic construct
  source 1..200: mol_type = other DNA
ccacttttcaagttgataacggactagccttatttaacttgctatgctgttttgaatggttcccaaaacagcatagctctaaaacacagttcctgactacgaaagagactccgtatcgtaaagtgaccgacggtgttgtaactgacgaaattcactacctgtctgctatcgaagaaggcaactacgttatcgccctccgc

SEQ ID NO: 15 - skipped

SEQ ID NO: 16 - DNA, 28 bp, synthetic construct
  source 1..28: mol_type = other DNA
gggtcctgtctgaaatcgacatcgccgc

SEQ ID NO: 17 - protein, 2590 aa, Homo sapiens
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 18 - protein, 2590 aa, synthetic construct
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLGLRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 19 - protein, 2590 aa, synthetic construct
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSFLAADYSQLGLRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 20 - protein, 772 aa, synthetic construct
SSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 21 - protein, 652 aa, synthetic construct
SSSSESLSIIDVASDQNLFQTFIKEWRCKKRFSISLACEKIRGSGDDTLVVGLAVCWGGRDAYYFSLGGSGGLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPGGSGGQFSTSKDVLNKLKALHPLPGLILEWRRITNAITKVVFPLQREKCLNPFLGMERIYPVSQSHTATGRITFTEPNIQNVPRDFEIKMGGSGGMPFSISMRHAFVPFPGGSILAADYSQLGLRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDGGSGGCPIRGGFFILQLHDELLYEVAEEDVVQVAQIVKNEMESAVKLSVKLKVKVKIGASWGELKDFDV